SEQUENCE LISTING (11 sequences)

SEQ ID NO: 1 - protein, 527 aa, Aspergillus aculeatus
  SIGNAL 1..19
MLKASLLSFVAFTAQVAHAAFGITTSSSAYVIDTNAPNQLKFTVSRSSCDITSIIHYGTELQYSSQGSHIGSGLGSATVTATQSGDYIKVTCVTDTLTQYMVVHNGDPIIHMATYITAEPSIGELRFIARLNSDLLPNEEPFGDVSTTADGTAIEGSDVFLVGSETRSKFYSSERFIDDQRHCIAGDAHRVCMILNQYESSSGGPFHRDINSNNGGSYNALYWYMNSGHVQTESYRMGLHGPYSMYFSRSGTPSTSIDTSFFADLDIKGYVAASGRGKVAGTASGADSSMDWVVHWYNDAAQYWTYTSSSGSFTSPAMKPGTYTMVYYQGEYAVATSSVTVSAGSTTTKNISGSVKTGTTIFKIGEWDGQPTGFRNAANQLRMHPSDSRMSSWGPLTYTVGSSALTDFPMAVFKSVNNPVTIKFTATSAQTGAATLRIGTTLSFAGGRPQATINSYTGSAPAAPTNLDSRGVTRGAYRGLGEVYDVSIPSGTIVAGTNTITINVISGSSGDTYLSPNFIFDCVELFQ

SEQ ID NO: 2 - protein, 378 aa, Aspergillus aculeatus
  SIGNAL 1..20
MHLNTTLLVSLALGAASVLASPAPPAITAPPTAEEIAKRATTCTFSGSNGASSASKSKTSCSTIVLSNVAVPSGTTLDLTKLNDGTHVIFSGETTFGYKEWSGPLISVSGSDLTITGASGHSINGDGSRWWDGEGGNGGKTKPKFFAAHSLTNSVISGLKIVNSPVQVFSVAGSDYLTLKDITIDNSDGDDNGGHNTDAFDIGTSTYVTISGATVYNQDDCVAVNSGENIYFSGGYCSGGHGLSIGSVGGRSDNTVKNVTFVDSTIINSDNGVRIKTNIDTTGSVSDVTYKDITLTSIAKYGIVVQQNYGDTSSTPTTGVPITDFVLDNVHGSVVSSGTNILISCGSGSCSDWTWTDVSVSGGKTSSKCTNVPSGASC

SEQ ID NO: 3 - protein, 238 aa, Aspergillus aculeatus
  SIGNAL 1..14
MKLSLLSLATLASAASLQRRSDFCGQWDTATAGDFTLYNDLWGESAGTGSQCTGVDSYSGDTIAWHTSWSWSGGSSSVKSYANAALTFTPTQLNCISSIPTTWKWSYSGSSIVADVAYDTFLAETASGSSKYEIMVWLAALGGAGPISSTGSTIATPTIAGVNWKLYSGPNGDTTVYSFVADSTTESFSGDLNDFFTYLVDNEGVSDELYLTTLEAGTEPFTGSNAKLTVSEYSISIE

SEQ ID NO: 4 - protein, 241 aa, Aspergillus luchuensis
  SIGNAL 1..15
MKVLALSALLSLASAASISRRSDFCDQWGTATAGDFILYNDLWGEDNASSGSQCTGVDSASGNEIAWHTSWSWEGGSSDVKSYANAALQFTGTQLSSISSIPSTWKWTYSGSDIVADVAYDMFLGSTADASSDEYEIMVWLAALGGAGPISSTGSTIATPTINGVTWDLYTGPNGDTTVYSFVAQSTTEDFSGDLNDFFTYLVDNEGVSDSLYLTTLEAGTEPFTGSNAELKVSEYSVSIE

SEQ ID NO: 5 - protein, 348 aa, Cohnella sp-60555
  SIGNAL 1..32
MMFKRTVTGMMALLLVLALFVAQGSQPHQAAAAPSFAKGADISWVPGMEAQGYKWKDKNGVQRDILDILKNDYQINSVRIRVWVNPSSSYTNGYLNKDRAAALAKRAKAAGMSVMLTLHYSDSWADPGQQTKPAAWKSYTFQQLMDAVWNWTRDVMTTMQANGVTPDWVQIGNETNNGMLWDDGKASLSMKNYAWLVNTGNNAVKSISSSTKTIVHLANGYDNSLFVWNIGGLIANGATFDIIGMSLYPSASDWSAKVTQTIANANDMISRYGKPIMVTEIGMDYSQPSAAKSFVSDIKTKIRNLSGGKGQGVFYWEPEATPGYNGGYSMGAWQADMKPTIALEGFWN

SEQ ID NO: 6 - protein, 406 aa, Aspergillus tubingensis
  SIGNAL 1..18
MALYRNLFFLASLGLSNAAPSKAQRAPDSSLHARAVCTPTAGGSSSTDDVPAITEALSSCGNGGTIVFPEGSTYYLNSVLDLSSCSDCDIQVEGLLKFASDTDYWSGRTAMISVSDVDGLKLRSLTGSGVIDGNGQDAWDLFASDSSYSRPTLLYITGGSNLEISGLRQKNPPNVFNSVKGGATNVVFSNLKINANSKSDNPPKNTDGFDIGESTYVTITEVTVVNDDDCVAFKPSSNYVTVDTISCTGSHGISVGSLGKSSDDSVKNIYVTGATMINSTKAAGIKTYPSGGDHGTSTVSNVTFNDFTVDNSDYAFQIQSCYGEDDDYCEENPGNAKLTDIVVSSFSGTTSDKYDPVVANLDCGADGTCGISISGFDVKAPSGKSEVLCANTPSGLGVTCTSGASG

SEQ ID NO: 7 - protein, 406 aa, Aspergillus aculeatus
  SIGNAL 1..18
MAPHWTSLLLAAVGLVRAAPSPVQHSPDAVLRPRAVCTPTAGGSSSIDDVPAITKAIASCGNGGTIVIPEGTTYYLNSVLDFAGCTGCDFQVEGLLKFASNTDYWEGRTAMISISKINGLKLRSLTGSGVIDGNGQDAWDVFASDSSYARPTLLYITGGSNLEFSNFRQKNPPNVFNSVKGGATNVVFSKLRMDATSKSDNDPKNTDGFDIGESTYVTIADTTVKNDDDCVAFKPSSNYVTVDTITCTGSHGISVGSLGKSSSDTVKNIYVTGATMINSTKAAGIKTYPSGGSHGVSTVSNVTFNDFTIDNSDYAFQIQSCYGEDASYCTTYPGNAKLSGIKVTGFSGTTSSKYDPTVANLNCGSGGTCDVTISEWSVKAPSGKSQVLCANTPSSLGVTCTSGASG

SEQ ID NO: 8 - protein, 389 aa, Aspergillus aculeatus
  SIGNAL 1..19
MRTTISVVLAALLLGQVDAAGVTGKAFGFASGTTGGGSAAAATPSSLAQLKEWITDDVARTIMIDREWDFTGTEGTTTGKCCSTKTTTCPGGTSKGQAWIQDHCDDGTWISCTYDNAARSPLDVGSNKSIVGVGSKGVLKGKGLRVRGGNSNVIIQNIHITDLNPEYVWGGDALTLDGADKVWIDHNKFSLIGRQMIVSGWNKAGHVTISNNEFDGKTSWSAGCNGKHYWTLLLLGAEDYYTFAGNWLHDVSGRAPHMGTDYDASTIYFHGVNNYFQNVGGHAFDVDTNTRLLLEGNYFDTVTTPMTPDSLTKGGQLYTTSTVAAASGCTSQLGYICEWNRASGSGSWPDRTDATVLTEFAQFKQYLIGHVGVAEVPANVTAHAGVGRL

SEQ ID NO: 9 - protein, 530 aa, Penicillium oxalicum
  SIGNAL 1..20
MLASGILLASLALLGKGVEAAFGVTTASDSYTIDTGSTNPLRYRVNRSNCDINSIKFYGTELQYGKQGSHIGSGLGKATVTATQNGNYIKVTCETSTLTHYYVARNGDPIIYMATYTTAEPSIGELRYIARLNANVLPNEEPFGVASNIAGGTAIEGSDVFKVGSQTRSKFYSSERFIDDHRHCVSGSQHRVCMILNQYETSSGGPFHRDINTNNVGDYTGLYWYMNSGHVQTESFRTGLHGPYLMYFSRSGTPSSDIDTSFFADLGIKGYVAASGRGKVTGTASGADSSMQWVIHWYNDKAQYWTYTASGGKFTSPAMKPGTYTMIYYQGEYPVAKTSVSVSAGSTVNKDISGSVKTGKTIFRIGQWDGKPTGFLNSDSQLRMHPTDQRMSPWTSSQPFTVGSSPTSAFPMAIFSAVNSPVTIKFTASSSQAAAATLRIGTTLSFAGGRPQAIINGWKGPIPSAPTNLNSRGVTRGAYRGLGEVYDVTIPAGVIKAGNSNEITISVVSGSSGDQFLSPNFIFDCIELFA

SEQ ID NO: 10 - protein, 574 aa, Penicillium rubens
  SIGNAL 1..19
MQILLSAIVALGIAQTATAAPTGAVCTGTFSAISAADFVSNLHPGWNLGNTLDAIPDEGSWNNVPVVASTFDTVKDAGFKSVRLPVTWADHFIGSSPDWTVDPTWLDRVSDVVDMIVARGLYAIVDVHHDSWNWADVTQSGANLTMIEEKFYSLWYQIGTKLACKSNLVAFEPINEPPCNTAADGTKINKLNKIFLQAINDAGGFNSQRVVTLVGGAQDSIKTADWFVAPSGFSNPYAIQFHYYSPYDFIFSAWGKTIWGSDSDKSAVQSDFQRLRNTFPDVPLLLGEYDASPTNTEPAARWKYVDFLTKTAASLDISCVLWDNGLDHLDRTTGVWRDTNSLSIITKSTESTANSLPDSTEDASATTQSSSAYIFHQYGTDVTEQTLPFIFNGNKLSSISDSTGSTLSGTDYSVSGANIIFTASYLSKHLSSTTAPGVIATLTLKFSGGDTSPVIQIVQWKAPTLSSTSAVASSVSGSDLSIPITWGGLPTVAAVKALTTSGKYLVDDWTVSLGPIQQARATYSSQYNWDDSHVILTSSAISSVISAGESTVFTFEFFPRDNGVANAVNFTLTV

SEQ ID NO: 11 - protein, 27 aa, Bacillus clausii
MKKPLGKIVASTALLISVAFSSSIASA

(19) United States
(12) Patent Application Publication
(10) Pub. No.: US 2026/0354418 A1
(43) Pub. Date: Nov. 12, 2026

(54) Fiber-Degrading Enzymes for Animal Feed Comprising an Oil Seed Material

(71) Applicant: NOVOZYMES A/S, Bagsvaerd (DK)

(72) Inventors: Eduardo Antonio DELLA PIA, Bagsvaerd (DK); Larissa STAACK, Bagsvaerd (DK)

(73) Assignee: NOVOZYMES A/S, Bagsvaerd (DK)

(21) Appl. No.: 19/135,610

(22) PCT Filed: Dec. 8, 2023
(86) PCT No.: PCT/EP2023/084825

§ 371 Date: Jun. 4, 2025

(30) Foreign Application Priority Data
Dec. 8, 2022 (EP) 22212208.7

(51) Int. Cl. A23K 20/189; A23K 10/30

(52) U.S. Cl. CPC A23K 20/189; A23K 10/30; C12Y 302/01089; C12Y 402/0201; C12Y 402/02023

(57) ABSTRACT

The present invention relates to the use of a fiber-degrading enzyme in animal feed comprising an oil seed thereby improving the availability of nutrients from the feed and the nutritional value of the animal feed. The present invention further relates to an animal feed comprising a fiber-degrading enzyme and an oil seed material.

### REFERENCE TO SEQUENCE LISTING

This application contains a Sequence Listing in computer readable form. The computer readable form is incorporated herein by reference.

### FIELD OF THE INVENTION

This invention relates to the field of fiber-degrading enzymes in animal feed comprising an oil seed.

### BACKGROUND OF THE INVENTION

Oil seed materials make up to about 25% of monogastric diets. They are inexpensive, available on a large scale, and have a high protein content. However, Oil seed materials contain substances that are almost not digested by monogastric animals, as these animals lack the relevant enzymes in their digestive tracts for digesting the substances. If a feed ingredient is almost not digested, the energy contained in the feed ingredient is not fully utilized. In addition, undigested substances have an effect on the viscosity of the feed within the digestive tract. Enhanced viscosity will result in an impaired digestibility of other nutrients.

Maintaining a healthy gut is important in monogastric animal production, and together with environmental conditions, the diet is the key contributing factor affecting the microbiota composition.

There is a need in the art to improve nutritional value of an animal feed comprising an oil seed material. Furthermore, there is a need in the art to improve intestinal health of a monogastric animal fed with a feed comprising an oil seed material.

### SUMMARY OF THE INVENTION

The present invention relates to a method of improving nutritional value of an animal feed comprising an oil seed material.

The present invention further relates to a method of improving growth performance of an animal, comprising administrating to the animal a fiber-degrading enzyme, an animal feed or animal feed additive comprising a fiber-degrading enzyme; preferably the growth performance is the growth rate, the feed conversion ratio, and/or the body weight gain.

The invention is directed to a method of improving the nutritional value of an animal feed comprising an oil seed material, said method comprising adding a fiber-degrading enzyme to said animal feed. A further aspect is directed to a method of improving growth performance of an animal, comprising administrating to the animal a fiber-degrading enzyme, an animal feed or animal feed additive comprising a fiber-degrading enzyme; preferably the growth performance is the growth rate, the feed conversion ratio, and/or the body weight gain.

The present invention further relates to a method of generating a prebiotic in-situ in an oil seed based animal feed, said method comprising adding a fiber-degrading enzyme to said animal feed, including a method for in-situ production of prebiotics in monogastric animals.

The present invention further relates to a method of decreasing an insoluble pectin fraction in an oil seed based animal feed.

The present invention further relates to a method of improving intestinal health of a monogastric animal, said method comprising administrating an oil seed based animal feed to said animal wherein said animal feed comprises a fiber-degrading enzyme.

The present invention further relates to a method of causing a butyrogenic effect in a monogastric animal.

The present invention further relates to an animal feed comprising a fiber-degrading enzyme and an oil seed material, such as an animal feed comprising a fiber-degrading enzyme and an oil seed material wherein the feed comprises oil seed material in an amount of 10 to 500 g/kg feed and the fiber-degrading enzyme in an amount of 0.1 to 500 mg enzyme protein/kg of feed. A further aspect is directed to an animal feed additive comprising a fiber-degrading enzyme and one or more additional components selected from the group consisting of: one or more vitamins; one or more minerals; one or more amino acids; one or more phytogenics; one or more prebiotics; one or more organic acids; and one or more other feed ingredients. A related aspect is directed to an animal feed comprising the animal feed additive of the invention and an oil seed material.

The present invention further relates to use of a fiber-degrading enzyme in preparation of an enzyme-enriched animal feed.

The present invention further relates to use of a combination of a polypeptide having rhamno-galacturonan lyase activity and a polypeptide having galactanase activity in an animal feed or animal feed additive; or use of a combination of rhamnogalacturonan lyase and pectin lyase in an animal feed or animal feed additive; or use of a combination of endo-beta-1,4-galactanase and pectin lyase in an animal feed or animal feed additive; or use of a combination of xyloglucan-specific endo-beta-1,4-glucanase/endo-xyloglucanase and pectin lyase in an animal feed or animal feed additive.

The present invention further relates to an animal feed additive comprising a polypeptide having rhamno-galacturonan lyase activity and a polypeptide having galactanase activity; preferably an animal feed additive comprising a combination of rhamnogalacturonan lyase and endo-beta-1,4-galactanase; a combination of rhamnogalacturonan lyase and pectin lyase; a combination of endo-beta-1,4-galactanase and pectin lyase; or a combination of xyloglucan-specific endo-beta-1,4-glucanase/endo-xyloglucanase and pectin lyase; and an animal feed comprising the animal feed additive.

The present invention further relates to a method of improving the Average Metabolizable Energy of plant-based diet in a monogastric animal.

The present invention further relates to a polypeptide having xyloglucan-specific endo-1,4-beta-glucanase activity, particularly a polypeptide having xyloglucan-specific endo-1,4-beta-glucanase activity, selected from the group consisting of:
(a) a polypeptide having at least 99.7%, at least 99.8%, at least 99.9% or 100% sequence identity to SEQ ID NO: 3 or the mature polypeptide of SEQ ID NO: 3;
(b) a polypeptide derived from SEQ ID NO: 3 or a mature polypeptide of SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide having at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID NO: 4 or the mature polypeptide of SEQ ID NO: 4;
(d) a polypeptide derived from SEQ ID NO: 4 or a mature polypeptide of SEQ ID NO: 4 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(e) a polypeptide derived from the polypeptide of (a), (b), (c) or (d), wherein the N- and/or C-terminal end has been extended by addition of one or more amino acids, e.g., 1 or 2 or 3 or 4 or 5 or 6 or 7 or 8 or 9 or 10 amino acids; and
(f) a fragment of the polypeptide of (a), (b), (c), (d) or (e);
wherein the polypeptide has xyloglucan-specific endo-1,4-beta-glucanase activity.

The present invention further relates to a polypeptide having xylogalacturonase activity, particularly a polypeptide having xylogalacturonase activity, selected from the group consisting of:
(a) a polypeptide having at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID NO: 6 or the mature polypeptide of SEQ ID NO: 6;
(b) a polypeptide derived from SEQ ID NO: 6 or a mature polypeptide of SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide having at least 82%, at least 83%, at least 84%, at least 85%, at least 86%, at least 87%, at least 88%, at least 89%, at least 90%, at least 91%, 92%, at least 93%, at least 94%, at least 95%, at least 96%, at least 97%, at least 98%, at least 99%, or 100% sequence identity to SEQ ID NO: 7 or the mature polypeptide of SEQ ID NO: 7;
(d) a polypeptide derived from SEQ ID NO: 7 or a mature polypeptide of SEQ ID NO: 7 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(e) a polypeptide derived from the polypeptide of (a), (b), (c) or (d), wherein the N- and/or C-terminal end has been extended by addition of one or more amino acids, e.g., 1 or 2 or 3 or 4 or 5 or 6 or 7 or 8 or 9 or 10 amino acids; and
(f) a fragment of the polypeptide of (a), (b), (c), (d) or (e);
wherein the polypeptide has xylogalacturonase activity.

The present invention further relates to a polypeptide having rhamnogalacturonan lyase activity, particularly a polypeptide having rhamnogalacturonan lyase activity, selected from the group consisting of:
(a) a polypeptide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 9 or the mature polypeptide of SEQ ID NO: 9;
(b) a polypeptide derived from SEQ ID NO: 9 or a mature polypeptide of SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide derived from the polypeptide of (a) or (b), wherein the N- and/or C-terminal end has been extended by addition of one or more amino acids, e.g., 1 or 2 or 3 or 4 or 5 or 6 or 7 or 8 or 9 or 10 amino acids; and
(d) a fragment of the polypeptide of (a), (b) or (c);
wherein the polypeptide has rhamnogalacturonan lyase activity.

The present invention further relates to a polypeptide having xyloglucan-specific endo-b-1,4-glucanase/endo-xyloglucanase, particularly a polypeptide having xyloglucan-specific endo-b-1,4-glucanase/endo-xyloglucanase, selected from the group consisting of:
(a) a polypeptide having at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 10 or the mature polypeptide of SEQ ID NO: 10;
(b) a polypeptide derived from SEQ ID NO: 10 or a mature polypeptide of SEQ ID NO: 10 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide derived from the polypeptide of (a) or (b), wherein the N- and/or C-terminal end has been extended by addition of one or more amino acids, e.g., 1 or 2 or 3 or 4 or 5 or 6 or 7 or 8 or 9 or 10 amino acids; and
(d) a fragment of the polypeptide of (a), (b) or (c);
wherein the polypeptide has xyloglucan-specific endo-b-1,4-glucanase/endo-xyloglucanase activity.

The present invention further relates to a polynucleotide encoding the polypeptide of the present invention.

The present invention further relates to an animal feed additive comprising a polypeptide of the present invention.

The present invention further relates to an animal feed comprising a polypeptide of the present invention.

### Summary of the Sequence Listing

SEQ ID NO: 1 is a polypeptide with rhamnogalacturonan lyase activity from Aspergillus aculeatus.

SEQ ID NO: 2 is a polypeptide with endo-polygalacturonase activity from Aspergillus aculeatus.

SEQ ID NO: 3 is a polypeptide with xyloglucan-specific endo-1,4-beta-glucanase activity from Aspergillus aculeatus.

SEQ ID NO: 4 is a polypeptide with xyloglucan-specific endo-1,4-beta-glucanase activity from Aspergillus luchuensis.

SEQ ID NO: 5 is a polypeptide with galactanase activity from Cohnella sp-60555.

SEQ ID NO: 6 is a polypeptide with xylogalacturonase activity from Aspergillus tubingensis.

SEQ ID NO: 7 is a polypeptide with xylogalacturonase activity from Aspergillus aculeatus.

SEQ ID NO: 8 is a polypeptide with pectin lyase activity from Aspergillus aculeatus.

SEQ ID NO: 9 is a polypeptide with rhamnogalacturonan lyase activity from Penicillium oxalicum.

SEQ ID NO: 10 is a polypeptide with xyloglucan-specific endo-b-1,4-glucanase/endo-xyloglucanase activity from Penicillium rubens.

SEQ ID NO: 11 is the Bacillus clausii secretion signal.

### DETAILED DESCRIPTION OF THE INVENTION

### Methods for Improving the Nutritional Value of an Animal Feed

The invention relates to the use of a fiber-degrading enzyme in animal feed comprising an oil seed thereby improving the availability of nutrients from the feed and the nutritional value of the animal feed.

According to the invention, supplementing an animal feed with a fiber-degrading enzyme (preferably a pectinase) gives an additional performance benefit in animals compared to the same animal feed but without the pectinase present. In a preferred embodiment, an animal feed comprises an oil seed material.

In a further aspect, the invention relates to a method of improving the nutritional value of an animal feed comprising an oil seed material, comprising adding a fiber-degrading enzyme to said animal feed.

In a preferred embodiment, the improvement is compared to the same animal feed or animal feed additive but excluding the fiber-degrading enzyme.

The term improving the nutritional value of an animal feed means improving the availability of nutrients in the feed. The nutritional values refer in particular to improving the solubilization and degradation of fibers in the oil seed materials, thereby increasing the amount of oligomers containing fucose, rhamnose, arabinose, galactose, glucose, xylose, glucuronic acid and/or galacturonic acid released which can be utilized by the animal and the animal microbiota. Consequently, an improved release of oligomers containing fucose, rhamnose, arabinose, galactose, glucose, xylose and/or galacturonic acid will result in an improvement of the nutritional value of the feed, thus resulting in increased the growth rate and/or the weight gain and/or the feed conversion (i.e., the weight of ingested feed relative to weight gain).

In a further aspect, the present invention relates to a method of improving growth performance of an animal, comprising administrating to the animal a fiber-degrading enzyme, an animal feed or animal feed additive comprising a fiber-degrading enzyme. In a preferred embodiment, the growth performance is the growth rate, the feed conversion ratio (FCR), and/or the body weight gain (BWG).

In a preferred embodiment, the improvement is compared to the animal fed with same animal feed or animal feed additive but excluding the fiber-degrading enzyme.

In one embodiment, the growth rate is improved by at least 1%, such as by at least 2%, at least 5% or at least 10%. In another embodiment, the growth rate is improved by between 1% and 15%, such as between 2% and 10%, between 4% and 8%, or any combination of these intervals.

In one embodiment, the FCR is improved by at least 1%, such as by at least 1.0%, at least 1.5% or at least 2.0%. In another embodiment, the FCR is improved by between 1% and 5%, such as between 1.5% and 4%, between 2% and 3%, or any combination of these intervals.

In one embodiment, the BWG is improved by at least 1%, such as by at least 1.0%, at least 1.5% or at least 2.0%. In another embodiment, the BWG is improved by between 1% and 5%, such as between 1.5% and 4%, between 2% and 3%, or any combination of these intervals.
Growth rate: growth rate of an animal can be measured by, for example, percent body weight increase/day.
Feed Conversion Ratio (FCR): FCR is a measure of an animal's efficiency in converting feed mass into increases of the desired output. Animals raised for meat—such as swine, poultry and fish—the output is the mass gained by the animal. Specifically, FCR is calculated as feed intake divided by body weight gain, all over a specified period. Improvement in FCR means reduction of the FCR value. A FCR improvement of 2% means that the FCR was reduced by 2%.
Body Weight Gain (BWG): “body weight gain” means an increase in live weight of an animal during a given period of time, e.g., the increase in weight from day 1 to day 21.

In particular embodiments, the oil seed material is selected from the group consisting of soybean, rapeseed, sunflower, peas, lupin, tepary bean, scarlet runner bean, slimjim bean, lima bean, French bean, Broad bean (fava bean), chickpea, lentil, peanut, linseed, cottonseed, or the combination thereof. In particular embodiments, the oil seed material is processed to be a processed form such as oil seed meal, full fat oil seed meal, oil seed protein concentrate, fermented oil seed meal or any combination thereof. In a preferred embodiment, the oil seed material is selected from the group consisting of soybean meal, rapeseed meal, sunflower meal, peas meal, peanut meal, linseed meal, cottonseed meal, or the combination thereof. In a further preferred embodiment, the oil seed material is selected from the group consisting of rapeseed meal and soybean meal. Rapeseed meal and soybean meal are by-products of bioethanol and food production. In one embodiment, the plant-based material is from the taxonomic subclass rosids. In one aspect, the plant-based material is from the taxonomic order Fabales, such as the family Fabaceae, preferably the subfamilies Caesalpinioideae or Mimosoideae or Papilionoideae, or more preferably from the tribes Phaseoleae, Cicereae, Genisteae, Fabeae, Dalbergieae or Phaseoleae. In one aspect, the plant-based material is from the taxonomic order Brassicales, such as the family Brassicaceae, preferably the tribe Brassiceae, more preferably the family Brassica.

The term “animal” refers to all animals including humans. Examples of animals are non-ruminants, and ruminants. Ruminant animals include, for example, animals such as sheep, goats, cattle, e.g., beef cattle, cows, and young calves, deer, yank, camel, llama and kangaroo. Non-ruminant animals include mono-gastric animals, e.g., pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), young calves; fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

In a preferred embodiment, the animal is a monogastric animal, preferably, the monogastric animal is selected from the group consisting of pigs or swine (including, but not limited to, piglets, growing pigs, and sows); poultry such as turkeys, ducks and chicken (including but not limited to broiler chicks, layers).

In a further aspect, the present invention relates to a method of generating a prebiotic in-situ in an oil seed based animal feed comprising adding a fiber-degrading enzyme to said animal feed.

Prebiotics are substances that induce the growth or activity of microorganisms (e.g., bacteria and fungi) that contribute to the well-being of their host. Prebiotics are typically non-digestible fiber compounds that pass undigested through the upper part of the gastrointestinal tract and stimulate the growth or activity of advantageous microorganisms that colonize the large bowel by acting as substrate for them. Normally, prebiotics increase the number or activity of bifidobacteria and lactic acid bacteria in the gastrointestinal tract.

In a further aspect, the present invention relates to a method of decreasing an insoluble pectin fraction in an oil seed based animal feed comprising adding a fiber-degrading enzyme to said animal feed. In the present invention, the fiber-degrading enzyme degrades the insoluble pectin fraction of said oil seed based animal feed so as to generate prebiotic oligomers and polymers comprising pectin oligosaccharides.

### Methods of Improving Intestinal Health of a Monogastric Animal

In the present invention, the fiber-degrading enzyme of the present invention has a beneficial effect on the accumulation of short-chain fatty acids from the fermentation of oil seed material by caecal microbiota. In one embodiment, the accumulation of butyrate is increased by one, two, three or more times. Increased formation of butyrate may indicate a better health status of the gut, since butyrate is a well-known gut health promoting molecule with anti-inflammatory properties. In a further embodiment, the accumulation of acetate is increased by one, two, three or more times. In a further embodiment, the accumulation of propionic acid is increased by one, two, three or more times.

In a further aspect, the present invention relates to a method of improving intestinal health of a monogastric animal comprising administrating an oil seed based animal feed to said animal, wherein said animal feed comprises a fiber-degrading enzyme. In one embodiment, the abundance of Enterococcus and Escherichia/Shigella in the fermentation of rapeseed meal (RSM) by chicken caecal microbiota drops in the presence of the fiber-degrading enzymes of the present invention. In a further embodiment, the enzymatic treatments favour the growth of Bacteroides, a genus that contains well known pectin-degrading bacteria that, in a cross-feeding manner, benefits the growth of butyrate producers. In a further embodiment, the relative abundance of Bacteroides increases 0.5%-100%, preferably 1-50%, more preferably 2-20% in the presence of the fiber-degrading enzymes of the present invention, compared to the control treatment. In a particular embodiment, the relative abundance of Bacteroides increases 5% in the presence of the galactanase, and, when combined with the rhamnogalacturonan endolyase (RG-I lyase), the relative abundance increases 10%, compared to the control treatment. In a further embodiment, Propionibacterium, genus known for its propionic acid producers, has its relative abundance increased 5%-2000%, preferably 10-1000%, more preferably 20-500% in the presence of the fiber-degrading enzymes of the present invention. In a particular embodiment, Propionibacterium, has its relative abundance doubled when the RG-I lyase was added to the galactanase, compared to the galactanase alone. In a further embodiment, the genus Lactobacillus, which hosts the most common probiotics, has its relative abundance increased 5%-2000%, preferably 10-1000%, more preferably 20-500% in the presence of the fiber-degrading enzymes of the present invention. In a particular embodiment, the genus Lactobacillus, has its relative abundance increased from 1.9% to 2.7% when RSM is treated with galactanase, and further increases to 4.5% when the RG-I lyase is combined with the galactanase. In a further embodiment, the relative abundance of the genera Butyricicoccus increases 1-5000%, preferably 5-2000%, more preferably 10-1000% in the presence of the fiber-degrading enzymes of the present invention, compared to the control treatment. In a particular embodiment, the relative abundance of the genera Butyricicoccus increases 40% in the presence of the galactanase, and it nearly triples when combined with the RG-1 lyase, compared to the control treatment.

In a further aspect, the present invention relates to a method for the in-situ production of prebiotics in monogastric animals comprising administrating an enzyme-enriched oil seed based animal feed to said animal wherein said animal feed comprises a fiber-degrading enzyme. In a further embodiment, the cecal butyrate levels in situ in said animal is increased. In a further embodiment, the microbiota composition in said animal is altered.

In a further embodiment, the present invention relates to a method of causing a butyrogenic effect in a monogastric animal comprising administrating an oil seed based animal feed to said animal, wherein said animal feed comprises a fiber-degrading enzyme.

### The Fiber-Degrading Enzyme of the Invention

Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB) and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes in families based on amino acid sequence similarities has been proposed a few years ago. They currently fall into 90 different families: See the CAZy(ModO) internet site (Coutinho, P. M. & Henrissat, B. (1999) Carbohydrate-Active Enzymes server at: http://afmb.cnrs-mrs.fr/~cazy/CAZY/index.html (corresponding papers: Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12; Coutinho, P. M. & Henrissat, B. (1999) The modular structure of cellulases and other carbohydrate-active enzymes: an integrated database approach. In “Genetics, Biochemistry and Ecology of Cellulose Degradation”, K. Ohmiya, K. Hayashi, K. Sakka, Y. Kobayashi, S. Karita and T. Kimura eds., Uni Publishers Co., Tokyo, pp. 15-23).

The term “fiber degrading enzyme” as used herein may include one or more of the following fiber-degrading enzymes selected from the group consisting of a pectinase, xyloglucan specific endo 1,4-beta glucanase, xyloglucan-specific endo-beta-1,4-glucanase/endo-xyloglucanase, and the combination thereof. In a preferred embodiment, the fiber-degrading enzyme is a pectinase. In a preferred embodiment, the fiber-degrading enzyme is selected from the group consisting of a pectinase, xyloglucan specific endo 1,4-beta glucanase, xyloglucan-specific endo-beta-1,4-glucanase/endo-xyloglucanase, and the combination thereof; preferably the fiber-degrading enzyme is a pectinase; more preferably the fiber-degrading enzyme is one or more pectinases selected from the group consisting of rhamnogalacturonan lyase, endo-beta-1,4-galactanase, polygalacturonase, rhamnogalacturonanase, pectin methyl esterase, pectin lyase, pectin acetyl esterase, galactan endo-beta-1,3-galactanase, xylogalacturonase, and the combination thereof. In a more preferred embodiment, the fiber-degrading enzyme is one or more pectinases selected from the group consisting of more selected from the group consisting of rhamnogalacturonan lyase (alpha-L-rhamnopyranosyl-1,4-alpha-D-galactopyranosyluronate endolyase, EC 4.2.2.23), endo-beta-1,4-galactanase (EC 3. 2. 1. 89), polygalacturonase (1,4-alpha-D-galacturonan glycanohydrolase, EC 3.2.1.15 and EC 3.2.1.67), rhamnogalacturonanase (rhamnogalacturonan alpha-D-galacturonic acid-1,2-alpha-L-rhamnose hydrolase, EC 3.2.1.171), pectin methyl esterase (pectin pectyl hydrolase, EC 3.1.1.11), pectin lyase ((1,4)-6-O-methyl-alpha-D-galacturonan lyase, EC 4.2.2.10), pectin acetyl esterase (acetic ester acetylhydrolase, EC 3.1.1.6), galactan endo-beta-1,3-galactanase (EC 3.2.1.181), xylogalacturonase, and the combination thereof. In a further preferred embodiment, the fiber-degrading enzyme is a pectinase selected from the group consisting of rhamnogalacturonan lyase, endo-beta-1,4-galactanase, pectin lyase and the combination thereof; preferably, the fiber-degrading enzyme is selected from the group consisting of a combination of rhamnogalacturonan lyase and endo-beta-1,4-galactanase; a combination of rhamnogalacturonan lyase and pectin lyase; a combination of endo-beta-1,4-galactanase and pectin lyase; and a combination of xyloglucan-specific endo-beta-1,4-glucanase/endo-xyloglucanase and pectin lyase.

Pectinase: The term “pectinase” is defined as a broad class of enzymes, which catalyzes hydrolysis of pectin, a structural plant cell wall acidic heteropolysaccharide with a backbone that contains 1,4-linked alpha-D-galactosyluronic acid residues. What follows is a definition of different classes of pectinases of the present invention. Activity units are defined for each of the pectinase classes. The skilled artisan can readily determine whether a polypeptide has a particular type of pectinase activity using the definitions and activity units below.

Rhamnogalacturonan Lyase: The term “rhamnogalacturonan lyase” (alpha-L-rhamnopyranosyl-1,4-alpha-D-galactopyranosyluronate endolyase, EC 4.2.2.23) is defined as an enzyme, which catalyzes endotype eliminative cleavage of L-alpha-rhamnopyranosyl-1,4-alpha-D-galactopyranosyluronic acid bonds of rhamnogalacturonan leaving L-rhamnopyranose at the reducing end and 4-deoxy-4,5-unsaturated D-galactopyranosyluronic acid at the non-reducing end. The rhamnogalacturonan lyase unit activity is defined as the amount of enzyme that produces 1 pmol of oligogalacturonides per minute, equivalent to the absorbance of 1 μmol unsaturated digalacturonide, using a molecular extinction coefficient for the dimer of 4600 M⁻¹ cm⁻¹ at 235 nm from rhamnogalacturonan under standard reaction conditions of pH 9.0, 37 °C, reaction buffer: 25 mM Tris/HCl, 25 mM glycine/NaOH, reaction time: 5 minutes.

Endo-beta-1,4-galactanase: The term “Endo-beta-1,4-galactanase (EC 3. 2. 1. 89)” is defined as an enzyme, which specifically hydrolyses (1->4)-beta-D-galactosidic linkages in type I arabinogalactans. Galactanase activity can be determined by reducing ends using the colorimetric assay developed by Lever (Analytical Biochemistry 47, 273-279, 1972). The galactanase produces reducing end sugars which react with PAHBAH generating an increase of colour which is proportional to the enzyme activity under the conditions used in the assay.

Polygalacturonase: The term “polygalacturonase” (1,4-alpha-D-galacturonan glycanohydrolase, EC 3.2.1.15 and EC 3.2.1.67) is defined as an enzyme, which catalyzes the hydrolysis of 1,4-alpha-D-galactosiduronic linkages in pectate and other galacturonans. Polygalacturonases are classified as either endo-polygalacturonases or exo-polygalacturonases. Endo-polygalacturonases (EC 3.2.1.15) catalyze the random cleavage of pectic acid, whereas exo-polygalacturonases (EC 3.2.1.67) catalyze the cleavage of pectic acid in a sequential manner on non-reducing ends of pectic acid producing either mono-galacturonate or di-galacturonate. Classes of polygalacturonases are differentiated by their characteristic amino acid sequences with commonly conserved, functional domain motifs known as SPNTDG (PG I), GDDC (PG II), CGPGHGISIGSLG (PG III), and RIK (PG IV). The polygalacturonase unit (PGU) is defined as the amount of enzyme, which will liberate 1.0 micromole galacturonic acid from poly-galacturonic acid per hour under the standard conditions pH 4.0, 25 °C (Kertesz, Z. I. (1955) Methods in Enzymology. 1, 162-164).

Rhamnogalacturonanase: The term “rhamnogalacturonanase” (rhamnogalacturonan alpha-D-galacturonic acid-1,2-alpha-L-rhamnose hydrolase, EC 3.2.1.171) is defined as an enzyme, which catalyzes the endohydrolysis of alpha-D-galacturonic acid-1,2-alpha-L-rhamnose glycosidic bond in the rhamnogalacturonan backbone with initial inversion of anomeric configuration releasing oligosaccharides with beta-D-galacturonic acid at the reducing end. Classes of rhamnogalacturonanases are differentiated by their specificity toward rhamnogalacturonan I (RG I) pectic heteropolysaccharides or rhamnogalacturonan II (RG II) pectic heteropolysaccharides. The rhamnogalacturonase activity unit (RGU) is defined as the amount of dye released, as measured by the absorbance change, from a solution of 20 mg/mL AZ-rhamnogalacturonan per mg enzyme per minute under standard reaction conditions pH 4.5, 40 °C, buffer: 25 mM sodium acetate, reaction time: 16 hours (de Vries, R. P. (2015) Biotechnology for Biofuels. 8:107).

Pectin Methyl Esterase: The term “pectin methylesterase” (pectin pectyl hydrolase, EC 3.1.1.11) is defined as an enzyme, which catalyzes demethoxylation of methyl ester groups in pectin chains to form pectate and releasing methanol. The pectinesterase unit (PMU) is defined as the amount of methanol liberated from a 1.0% solution of pectin containing 0.1 M sodium chloride in 30 minutes per gram of enzyme under the standard conditions pH 7.5, 30 °C (Kertesz, Z. I. (1955) Methods in Enzymology. 1, 162-164).

Pectin Lyase: The term “pectin lyase” ((1,4)-6-O-methyl-alpha-D-galacturonan lyase, EC 4.2.2.10) is defined as an enzyme, which catalyzes the eliminative cleavage of 1,4-alpha-D-galacturonan methyl esters to oligosaccharides with 4-deoxy-6-O-methyl-alpha-D-galact-4-enuronosyl groups at the non-reducing ends. The pectin lyase unit (PLU) is defined as the amount of enzyme, which will result in a change in absorbance of 1.0 at 235 nm in a solution of 0.5% w/v pectin under the standard conditions of pH 6.0, 40 °C, reaction buffer: 100 mM citric acid, 100 mM sodium phosphate, reaction time: 5 minutes (Albersheim, P. (1966) Methods in Enzymology, Vol. 8, 628-631).

Pectin Acetyl Esterase: The term “pectin acetylesterase” (acetic ester acetylhydrolase, EC 3.1.1.6) is defined as an enzyme, which catalyzes deacetylation of acetyl ester groups in pectin chains to form pectate and releasing acetic acid. The pectin acetylesterase activity unit is defined as the amount of p-nitrophenol in mmol as measured by absorbance at 460 nm released from a 2 mM solution of p-nitrophenol-acetyl by 1 mg of enzyme in 1 minute under standard assay conditions pH 7.4, 37 °C, reaction buffer: 25 mm Tris-HCl, 50 mm EDTA, and 150 mm MgCl₂, reaction time: 1 minute (Pogorelko, G. (2013) BIOCHEMISTRY AND METABOLISM. 162: 9-23).

Galactan endo-beta-1,3-galactanase: The term “galactan endo-beta-1,3-galactanase, (EC 3.2.1.181)” is defined as an enzyme, which catalyzes the endohydrolysis of beta-1,3 bonds in arabinogalactan requiring at least three continuous beta-1,3-residues. The beta-galactanase activity unit is defined as the amount of enzyme that releases 1 pmol of galactose from a 1% solution of beta-galactan per minute under standard reaction conditions pH 4.0, 37 °C, reaction buffer: 100 mM sodium acetate/acetic acid with 0.2% bovine serum albumin, reaction time: 4 hours (Carey, A. T. (1995) Plant Physiol. 108: 1099-1107).

Xylogalacturonase: The term “xylogalacturonase” is defined as an enzyme, which has the ability to cleave a galacturonic acid polymer (for example as found in pectin) which may be at least partially substituted with xylose at internal glycosidic bonds.

Xyloglucan specific endo 1,4-beta glucanase: The term “xyloglucan specific endo 1,4-beta glucanase” is defined as an enzyme that catalyzes the chemical reaction

xyloglucan+H2O⇄xyloglucan oligosaccharides

Xyloglucan-specific endo-beta-1,4-glucanaselendo-xyloglucanase: The term “Xyloglucan-specific endo-beta-1,4-glucanase/endo-xyloglucanase” is defined as an enzyme, which has the activity of endo-β-1,4-glucanase, endo-β-1,4-xylanase, endo-β-1,3-1,4-glucanase, xyloglucan-specific endo-β-1,4-glucanase/endo-xyloglucanase.

In a further embodiment, the fiber-degrading enzyme is rhamnogalacturonan lyase (alpha-L-rhamnopyranosyl-1,4-alpha-D-galactopyranosyluronate endolyase, EC 4.2.2.23).

In a further embodiment, the fiber-degrading enzyme is endo-beta-1,4-galactanase (EC 3.2. 1.89).

In an embodiment, the fiber-degrading enzyme is polygalacturonase (1,4-alpha-D-galacturonan glycanohydrolase, EC 3.2.1.15 and EC 3.2.1.67).

In a further embodiment, the fiber-degrading enzyme is rhamnogalacturonanase (rhamnogalacturonan alpha-D-galacturonic acid-1,2-alpha-L-rhamnose hydrolase, EC 3.2.1.171).

In a further embodiment, the fiber-degrading enzyme is pectin methyl esterase (pectin pectyl hydrolase, EC 3.1.1.11).

In a further embodiment, the fiber-degrading enzyme is pectin lyase ((1,4)-6-O-methyl-alpha-D-galacturonan lyase, EC 4.2.2.10).

In a further embodiment, the fiber-degrading enzyme is pectin acetyl esterase (acetic ester acetylhydrolase, EC 3.1.1.6).

In a further embodiment, the fiber-degrading enzyme is galactan endo-beta-1,3-galactanase (EC 3.2.1.181).

In a further embodiment, the fiber-degrading enzyme is xylogalacturonase.

In a further embodiment, the fiber-degrading enzyme is xyloglucan specific endo 1,4-beta glucanase.

In a further embodiment, the fiber-degrading enzyme is xyloglucan-specific endo-beta-1,4-glucanase/endo-xyloglucanase.

In a further embodiment, the fiber-degrading enzyme is a combination of a pectinase and xyloglucan specific endo 1,4-beta glucanase.

In a further embodiment, the fiber-degrading enzyme is a combination of a pectinase and xyloglucan-specific endo-beta-1,4-glucanase/endo-xyloglucanase.

In a further embodiment, the fiber-degrading enzyme is a combination of a xyloglucan specific endo 1,4-beta glucanase and xyloglucan-specific endo-beta-1,4-glucanase/endo-xyloglucanase.

In a further embodiment, the fiber-degrading enzyme is a combination of rhamnogalacturonan lyase, and endo-beta-1,4-galactanase.

In a further embodiment, the fiber-degrading enzyme is a combination of rhamnogalacturonan lyase and polygalacturonase.

In a further embodiment, the fiber-degrading enzyme is a combination of rhamnogalacturonan lyase and rhamnogalacturonanase.

In a further embodiment, the fiber-degrading enzyme is a combination of rhamnogalacturonan lyase and pectin methyl esterase.

In a further embodiment, the fiber-degrading enzyme is a combination of rhamnogalacturonan lyase and pectin lyase.

In a further embodiment, the fiber-degrading enzyme is a combination of rhamnogalacturonan lyase, and pectin acetyl esterase.

In a further embodiment, the fiber-degrading enzyme is a combination of rhamnogalacturonan lyase and galactan endo-beta-1,3-galactanase.

In a further embodiment, the fiber-degrading enzyme is a combination of rhamnogalacturonan lyase and xylogalacturonase.

In a further embodiment, the fiber-degrading enzyme is a combination of endo-beta-1,4-galactanase and polygalacturonase.

In a further embodiment, the fiber-degrading enzyme is a combination of endo-beta-1,4-galactanase and rhamnogalacturonanase.

In a further embodiment, the fiber-degrading enzyme is a combination of endo-beta-1,4-galactanase, and pectin methyl esterase.

In a further embodiment, the fiber-degrading enzyme is a combination of endo-beta-1,4-galactanase and pectin lyase.

In a further embodiment, the fiber-degrading enzyme is a combination of endo-beta-1,4-galactanase and pectin acetyl esterase.

In a further embodiment, the fiber-degrading enzyme is a combination of endo-beta-1,4-galactanase and galactan endo-beta-1,3-galactanase.

In a further embodiment, the fiber-degrading enzyme is a combination of endo-beta-1,4-galactanase, and xylogalacturonase.

In a further embodiment, the fiber-degrading enzyme is a combination of polygalacturonase and rhamnogalacturonanase.

In a further embodiment, the fiber-degrading enzyme is a combination of polygalacturonase and pectin methyl esterase.

In a further embodiment, the fiber-degrading enzyme is a combination of polygalacturonase and pectin acetyl esterase.

In a further embodiment, the fiber-degrading enzyme is a combination of polygalacturonase and galactan endo-beta-1,3-galactanase.

In a further embodiment, the fiber-degrading enzyme is a combination of polygalacturonase and xylogalacturonase.

In a further embodiment, the fiber-degrading enzyme is a combination of rhamnogalacturonanase and pectin methyl esterase.

In a further embodiment, the fiber-degrading enzyme is a combination of rhamnogalacturonanase and pectin lyase.

In a further embodiment, the fiber-degrading enzyme is a combination of rhamnogalacturonanase and pectin acetyl esterase.

In a further embodiment, the fiber-degrading enzyme is a combination of rhamnogalacturonanase and galactan endo-beta-1,3-galactanase.

In a further embodiment, the fiber-degrading enzyme is a combination of rhamnogalacturonanase and xylogalacturonase.

In a further embodiment, the fiber-degrading enzyme is a combination of pectin methyl esterase, and pectin lyase.

In a further embodiment, the fiber-degrading enzyme is a combination of pectin methyl esterase and pectin acetyl esterase.

In a further embodiment, the fiber-degrading enzyme is a combination of pectin methyl esterase and galactan endo-beta-1,3-galactanase.

In a further embodiment, the fiber-degrading enzyme is a combination of pectin methyl esterase, and xylogalacturonase.

In a further embodiment, the fiber-degrading enzyme is a combination of pectin lyase and pectin acetyl esterase.

In a further embodiment, the fiber-degrading enzyme is a combination of pectin lyase and galactan endo-beta-1,3-galactanase.

In a further embodiment, the fiber-degrading enzyme is a combination of pectin lyase and xylogalacturonase.

In a further embodiment, the fiber-degrading enzyme is a combination of pectin acetyl esterase and galactan endo-beta-1,3-galactanase.

In a further embodiment, the fiber-degrading enzyme is a combination of pectin acetyl esterase and xylogalacturonase.

In a further embodiment, the fiber-degrading enzyme is a combination of galactan endo-beta-1,3-galactanase, and xylogalacturonase.

### Sources of the Fiber-Degrading Enzyme

The fiber-degrading enzyme of the present invention may be obtained from microorganisms of any genus. For purposes of the present invention, the term “obtained from” as used herein in connection with a given source shall mean that the enzyme encoded by a polynucleotide is produced by the source or by a strain in which the polynucleotide of the invention has been inserted. In one aspect, the enzyme obtained from a given source is secreted extracellularly.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

The enzymes may be identified and obtained from other sources including microorganisms isolated from nature (e.g., soil, composts, water, etc.) or DNA samples obtained directly from natural materials (e.g., soil, composts, water, etc.) using the abovementioned probes. Techniques for isolating microorganisms and DNA directly from natural habitats are well known in the art. A polynucleotide encoding the enzyme may then be obtained by similarly screening a genomic DNA or cDNA library of another microorganism or mixed DNA sample. Once a polynucleotide encoding an enzyme has been detected with the probe(s), the polynucleotide can be isolated or cloned by utilizing techniques that are known to those of ordinary skill in the art (see, e.g., Davis et al., 2012, Basic Methods in Molecular Biology, Elsevier).

In one embodiment, the fiber-degrading enzyme is obtained from or obtainable from Aspergillus or Penicillium or Cohnella.

In one embodiment, the fiber-degrading enzyme is a rhamnogalacturonan lyase obtained from an Aspergillus or Penicillium, e.g., a rhamnogalacturonan lyase obtained from A. aculeatus or Penicillium oxalicum.

In one embodiment, the fiber-degrading enzyme is an endo-polygalacturonase obtained from Aspergillus, e.g., an endo-polygalacturonase obtained from A. aculeatus.

In one embodiment, the fiber-degrading enzyme is a xyloglucan-specific endo-1,4-beta-glucanase obtained from or obtainable from Aspergillus, e.g., a xyloglucan-specific endo-1,4-beta-glucanase obtained from A. aculeatus or A. luchuensis.

In one embodiment, the fiber-degrading enzyme is a galactanase obtained from or obtainable from Cohnella, e.g., a galactanase obtained from Cohnella sp-60555.

In one embodiment, the fiber-degrading enzyme is a xylogalacturonase obtained from or obtainable from Aspergillus, e.g., a xylogalacturonase obtained from Aspergillus tubingensis or Aspergillus aculeatus.

In one embodiment, the fiber-degrading enzyme is a pectin lyase obtained from or obtainable from Aspergillus, e.g., a pectin lyase obtained from Aspergillus aculeatus.

In one embodiment, the fiber-degrading enzyme is an endo-beta-1,4-glucanase/endo-xyloglucanase obtained from or obtainable from Penicillium, e.g., an endo-beta-1,4-glucanase/endo-xyloglucanase obtained from Penicillium rubens.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of the abovementioned bacteria and fungi are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

Questions relating to taxonomy can be solved by consulting a taxonomy data base, such as the NCBI Taxonomy Browser which is available at the following internet site: http://www.ncbi.nlm.nih.gov/Taxonomy/taxonomyhome.html/. However, preferably reference is had to the following handbooks: Dictionary of the Fungi, 9^{th} edition, edited by Kirk, P. M., P. F. Cannon, J. C. David & J. A. Stalpers, CAB Publishing, 2001; and Bergey's Manual of Systematic Bacteriology, Second edition (2005).

The term “a” as used herein in whatever context means “one or more”, preferably “at least one”. This is the case, e.g., for the use in claim 1 of “a” fiber-degrading enzyme as specified, which is considered equivalent to claiming the use of “at least one” or “one or more” such fiber degrading enzyme.

The term “variant” refers to a fiber-degrading enzyme which comprises a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position. The variant may be a natural variant (allelic variant) or prepared synthetically. Preferably, amino acid changes are of a minor nature, e.g., conservative amino acid substitutions that do not significantly affect the folding and/or activity of the protein; small deletions; small amino- or carboxyl-terminal extensions, such as an amino-terminal methionine residue; a small linker peptide; or a small extension that facilitates purification by changing net charge or another function, such as a poly-histidine tract, an antigenic epitope or a binding domain.

A “fragment” of a specified fiber-degrading enzyme has one or more amino acids deleted from the amino and/or carboxyl terminus of the amino acid sequence of the fiber degrading enzyme.

For purposes of the above definitions of variant and fragment, the term “small” as well as the term “one or more” refer to a maximum of 30 changes (for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) as compared to the specified fiber degrading enzyme. In preferred embodiments of either of these definitions, the number of changes is below 30, 25, 20, 15, 10, or below 5.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., fiber-degrading enzyme activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest and can be applied to polypeptides of unknown structure.

Mature polypeptide: The term “mature polypeptide” means a polypeptide in its mature form following N-terminal and/or C-terminal processing (e.g., removal of signal peptide). In one embodiment, the mature polypeptide of the rhamnogalacturonan lyase of SEQ ID NO: 1 is amino acids 20-527 thereof; the mature polypeptide of the endo-polygalacturonase of SEQ ID NO: 2 is amino acids 21-378 thereof; the mature polypeptide of the xyloglucan-specific endo-1,4-beta-glucanase of SEQ ID NO: 3 is amino acids 15-238 thereof; the mature polypeptide of the xyloglucan-specific endo-1,4-beta-glucanase of SEQ ID NO: 4 is amino acids 16-241 thereof; the mature polypeptide of the galactanase of SEQ ID NO: 5 is amino acids 33-348 thereof; the mature polypeptide of the xylogalacturonase of SEQ ID NO: 6 is amino acids 19-406 thereof; the mature polypeptide of the xylogalacturonase of SEQ ID NO: 7 is amino acids 19-406 thereof; the mature polypeptide of the pectin lyase of SEQ ID NO: 8 is amino acids 20-389 thereof; the mature polypeptide of the rhamnogalacturonan lyase of SEQ ID NO: 9 is amino acids 21-530 thereof; or the mature polypeptide of the endo-β-1,4-glucanase/endo-xyloglucanase of SEQ ID NO: 10 is amino acids 20-574 thereof.

Mature polypeptide coding sequence: The term “mature polypeptide coding sequence” means a polynucleotide that encodes a mature polypeptide having a fiber-degrading enzyme activity.

In a particular embodiment the fiber-degrading enzyme of the invention is isolated, i.e., essentially free of other polypeptides of enzyme activity, e.g., at least about 20% pure, preferably at least about 40% pure, more preferably about 60% pure, even more preferably about 80% pure, most preferably about 90% pure, and even most preferably about 95% pure, as determined by SDS-PAGE. As it is generally known in the art, for detection purposes the SDS-gel can be stained with Coomassie or silver staining. It should be ensured that overloading has not occurred, e.g., by checking linearity by applying various concentrations in different lanes on the gel. Such polypeptide preparations are in particular obtainable using recombinant methods of production, whereas they are not so easily obtained and also subject to a much higher batch-to-batch variation when the polypeptide is produced by traditional fermentation methods.

The polypeptides comprised in the composition of the invention are preferably also purified. The term purified refers to a protein-enriched preparation, in which a substantial amount of low molecular components, typical residual nutrients and minerals originating from the fermentation, have been removed. Such purification can, e.g., be by conventional chromatographic methods such as ion-exchange chromatography, hydrophobic interaction chromatography and size exclusion chromatography (see, e.g., Protein Purification, Principles, High Resolution Methods, and Applications. Editors: Jan-Christer Janson, Lars Ryden, VCH Publishers, 1989).

The use of an isolated and/or purified polypeptide according to the invention is advantageous. For instance, it is much easier to correctly dose enzymes that are essentially free from interfering or contaminating other enzymes. The terms correctly dose refer in particular to the objective of obtaining consistent and constant animal feeding results, and the capability of optimizing dosage based upon the desired effect.

Sequence identity: The relatedness between two amino acid sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

In one embodiment, the fiber-degrading enzyme has at least 60% sequence identity to the mature polypeptide of the rhamnogalacturonan lyase of SEQ ID NO: 1. In another embodiment, the fiber-degrading enzyme has at least 60% sequence identity to the mature polypeptide of the endo-polygalacturonase of SEQ ID NO: 2. In another embodiment, the fiber-degrading enzyme has at least 60% sequence identity to the mature polypeptide of the xyloglucan-specific endo-1,4-beta-glucanase of SEQ ID NO: 3. In another embodiment, the fiber-degrading enzyme has at least 60% sequence identity to the mature polypeptide of the xyloglucan-specific endo-1,4-beta-glucanase of SEQ ID NO: 4. In another embodiment, the fiber-degrading enzyme has at least 60% sequence identity to the mature polypeptide of the galactanase of SEQ ID NO: 5. In another embodiment, the fiber-degrading enzyme has at least 60% sequence identity to the mature polypeptide of the xylogalacturonase of SEQ ID NO: 6. In another embodiment, the fiber-degrading enzyme has at least 60% sequence identity to the mature polypeptide of the xylogalacturonase of SEQ ID NO: 7. In another embodiment, the fiber-degrading enzyme has at least 60% sequence identity to the mature polypeptide of the pectin lyase of SEQ ID NO: 8. In another embodiment, the fiber-degrading enzyme has at least 60% sequence identity to the mature polypeptide of the rhamnogalacturonan lyase of SEQ ID NO: 9. In another embodiment, the fiber-degrading enzyme has at least 60% sequence identity to the mature polypeptide of the endo-β-1,4-glucanase/endo-xyloglucanase of SEQ ID NO: 10.

In particular embodiments, the degree of sequence identity is at least 65%, at least 70%, at least 75%, at least 80%, at least 85%, at least 90%, at least 95%, at least 97%, at least 99% or 100%. In further embodiments, the degree of sequence identity is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%.

In further particular embodiments, the fiber-degrading enzyme is selected from the group consisting of:
(a) a rhamnogalacturonan lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 1 or a mature polypeptide of SEQ ID NO: 1;
(b) a rhamnogalacturonan lyase derived from SEQ ID NO: 1 or a mature polypeptide of SEQ ID NO: 1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a rhamnogalacturonan lyase derived from (a), or (b), wherein the N- and/or C-terminal end has been extended by addition of one or more amino acids, e.g., 1 or 2 or 3 or 4 or 5 or 6 or 7 or 8 or 9 or 10 amino acids;
(d) a fragment of the rhamnogalacturonan lyase of (a), (b), or (c), having the rhamnogalacturonan lyase activity;
(e) an endo-polygalacturonase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 2 or a mature polypeptide of SEQ ID NO: 2;
(f) an endo-polygalacturonase derived from SEQ ID NO: 2 or a mature polypeptide of SEQ ID NO: 2 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(g) an endo-polygalacturonase derived from (e), or (f), wherein the N- and/or C-terminal end has been extended by addition of one or more amino acids, e.g., 1 or 2 or 3 or 4 or 5 or 6 or 7 or 8 or 9 or 10 amino acids;
(h) a fragment of the endo-polygalacturonase of (e), (f), or (g), having the endo-polygalacturonase activity;
(i) a xyloglucan-specific endo-1,4-beta-glucanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 3 or a mature polypeptide of SEQ ID NO: 3;
(j) a xyloglucan-specific endo-1,4-beta-glucanase derived from SEQ ID NO: 3 or a mature polypeptide of SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(k) a xyloglucan-specific endo-1,4-beta-glucanase derived from (i), or (j), wherein the N- and/or C-terminal end has been extended by addition of one or more amino acids, e.g., 1 or 2 or 3 or 4 or 5 or 6 or 7 or 8 or 9 or 10 amino acids;
(l) a fragment of the xyloglucan-specific endo-1,4-beta-glucanase of (i), (j), or (k), having the xyloglucan-specific endo-1,4-beta-glucanase activity;
(m) a xyloglucan-specific endo-1,4-beta-glucanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 4 or a mature polypeptide of SEQ ID NO: 4;
(n) a xyloglucan-specific endo-1,4-beta-glucanase derived from SEQ ID NO: 4 or a mature polypeptide of SEQ ID NO: 4 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(o) a xyloglucan-specific endo-1,4-beta-glucanase derived from (m), or (n), wherein the N- and/or C-terminal end has been extended by addition of one or more amino acids, e.g., 1 or 2 or 3 or 4 or 5 or 6 or 7 or 8 or 9 or 10 amino acids; and
(p) a fragment of the xyloglucan-specific endo-1,4-beta-glucanase of (m), (n), or (o), having a xyloglucan-specific endo-1,4-beta-glucanase activity;
(q) a galactanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 5 or a mature polypeptide of SEQ ID NO: 5;
(r) a galactanase derived from SEQ ID NO: 5 or a mature polypeptide of SEQ ID NO: 5 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(s) a galactanase derived from (q) or (r), wherein the N- and/or C-terminal end has been extended by addition of one or more amino acids, e.g., 1 or 2 or 3 or 4 or 5 or 6 or 7 or 8 or 9 or 10 amino acids;
(t) a fragment of the galactanase of (q), (r), or (s), having the galactanase activity;
(u) a xylogalacturonase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 6 or a mature polypeptide of SEQ ID NO: 6;
(v) a xylogalacturonase derived from SEQ ID NO: 6 or a mature polypeptide of SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(w) a xylogalacturonase derived from (u) or (v), wherein the N- and/or C-terminal end has been extended by addition of one or more amino acids, e.g., 1 or 2 or 3 or 4 or 5 or 6 or 7 or 8 or 9 or 10 amino acids;
(x) a fragment of the xylogalacturonase of (u), (v), or (w), having the xylogalacturonase activity;
(y) a xylogalacturonase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 7 or a mature polypeptide of SEQ ID NO: 7;
(z) a xylogalacturonase derived from SEQ ID NO: 7 or a mature polypeptide of SEQ ID NO: 7 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(aa) a xylogalacturonase derived from (y) or (z), wherein the N- and/or C-terminal end has been extended by addition of one or more amino acids, e.g., 1 or 2 or 3 or 4 or 5 or 6 or 7 or 8 or 9 or 10 amino acids;
(bb) a fragment of the xylogalacturonase of (y), (z) or (aa) having the xylogalacturonase activity;
(cc) a pectin lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 8 or a mature polypeptide of SEQ ID NO: 8;
(dd) a pectin lyase derived from SEQ ID NO: 8 or a mature polypeptide of SEQ ID NO: 8 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(ee) a pectin lyase derived from (cc) or (dd), wherein the N- and/or C-terminal end has been extended by addition of one or more amino acids, e.g., 1 or 2 or 3 or 4 or 5 or 6 or 7 or 8 or 9 or 10 amino acids;
(ff) a fragment of the pectin lyase of (cc), (dd), or (ee) having the pectin lyase activity;
(gg) a rhamnogalacturonan lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 9 or a mature polypeptide of SEQ ID NO: 9;
(hh) a rhamnogalacturonan lyase derived from SEQ ID NO: 9 or a mature polypeptide of SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(ii) a rhamnogalacturonan lyase derived from (gg) or (hh), wherein the N- and/or C-terminal end has been extended by addition of one or more amino acids, e.g., 1 or 2 or 3 or 4 or 5 or 6 or 7 or 8 or 9 or 10 amino acids; and
(jj) a fragment of the rhamnogalacturonan lyase of (gg), (hh) or (ii) having rhamnogalacturonan lyase activity;
(kk) a xyloglucan-specific endo-b-1,4-glucanase/endo-xyloglucanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 10 or a mature polypeptide of SEQ ID NO: 10;
(ll) a xyloglucan-specific endo-b-1,4-glucanase/endo-xyloglucanase derived from SEQ ID NO: 10 or a mature polypeptide of SEQ ID NO: 10 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(mm) a xyloglucan-specific endo-b-1,4-glucanase/endo-xyloglucanase derived from (kk) or (ll), wherein the N- and/or C-terminal end has been extended by addition of one or more amino acids, e.g., 1 or 2 or 3 or 4 or 5 or 6 or 7 or 8 or 9 or 10 amino acids; and
(nn) a fragment of the xyloglucan-specific endo-b-1,4-glucanase/endo-xyloglucanase of (kk), (ll) or (mm) having a xyloglucan-specific endo-b-1,4-glucanase/endo-xyloglucanase activity.

In still further particular embodiments, the fiber-degrading enzyme of the invention comprises (preferably has, or consists of) a mature polypeptide of any one of the fiber-degrading enzyme of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and/or SEQ ID NO: 10; or a variant or fragment thereof that has fiber-degrading enzyme activity.

### Animal Feed and Animal Feed Additive

The present invention is also directed to methods for using a fiber-degrading enzyme of the invention in preparation of an enzyme-enriched animal feed, as well as to animal feed and feed additives comprising a fiber-degrading enzyme of the invention.

The term “animal feed” refers to any compound, preparation, or mixture suitable for, or intended for intake by an animal. Animal feed for a mono-gastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

The term “concentrates” means feed with high protein and energy concentrations, such as fish meal, molasses, oligosaccharides, sorghum, seeds and grains (either whole or prepared by crushing, milling, etc., from, e.g., corn, oats, rye, barley, wheat), oilseed press cake (e.g., from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

In particular embodiments, the fiber-degrading enzyme of the invention is for use in feed for (i) non-ruminant animals; preferably (ii) mono-gastric animals; more preferably (iii) pigs, poultry, fish, and crustaceans; or, most preferably, (iv) pigs and poultry.

The fiber-degrading enzyme of the invention can be fed to the animal before, after, or simultaneously with the diet. The latter is preferred.

The term feed, feed composition, or diet means any compound, preparation, mixture, or composition suitable for, or intended for intake by an animal. More information about animal feed compositions is found below.

In one embodiment, the present invention relates to an animal feed comprising a fiber-degrading enzyme and an oil seed material wherein the feed comprises oil seed material in an amount of 10 to 500 g/kg feed and the fiber-degrading enzyme in an amount of 0.1 to 500 mg enzyme protein/kg of feed.

The dosage of the fiber-degrading enzyme of the invention can be optimized using simple trial-and-error methods as is known in the art. Different Pectinase may have different optimum dosage ranges. Examples of suitable dosage ranges are: 0.1-500 mg enzyme protein (EP)/kg diet (substrate); preferably 0.2-400, 0.5-300, 1-200, or 2-100 mg EP/kg diet.

For determining mg enzyme protein of fiber-degrading enzyme per kg feed, the fiber-degrading enzyme is purified from the feed composition, and the specific activity of the purified fiber-degrading enzyme is determined using a relevant assay. The fiber-degrading enzyme activity of the feed composition as such is also determined using the same assay, and on the basis of these two determinations, the dosage in mg enzyme protein fiber-degrading enzyme per kg feed is calculated.

The same principles apply for determining mg enzyme protein fiber-degrading enzyme in feed additives. Of course, if a sample is available of the fiber-degrading enzyme used for preparing the feed additive or the feed, the specific activity is determined from this sample (no need to purify the fiber-degrading enzyme from the feed composition or the additive).

In a further aspect, the present invention relates to an animal feed additive, comprising a fiber-degrading enzyme and one or more additional components selected from the group consisting of: one or more vitamins; one or more minerals; one or more amino acids; one or more phytogenics; one or more prebiotics; one or more organic acids; and one or more other feed ingredients.

The following are non-exclusive lists of examples of these components: Examples of fat-soluble vitamins are vitamin A, vitamin D3, vitamin E, and vitamin K, e.g., vitamin K3.

Examples of water-soluble vitamins are vitamin B12, biotin and choline, vitamin B1, vitamin B2, vitamin B6, niacin, folic acid and panthothenate, e.g., Ca-D-panthothenate.

Examples of trace minerals are manganese, zinc, iron, copper, iodine, selenium, and cobalt.

Examples of macro minerals are calcium, phosphorus and sodium.

Examples of amino acids which are used in animal feed are lysine, alanine, beta-alanine, threonine, methionine and tryptophan.

Phytogenics are a group of natural growth promoters or non-antibiotic growth promoters used as feed additives, derived from herbs, spices or other plants. Phytogenics can be single substances prepared from essential oils/extracts, essential oils/extracts, single plants and mixture of plants (herbal products) or mixture of essential oils/extracts/plants (specialized products).

Examples of phytogenics are rosemary, sage, oregano, thyme, clove, and lemongrass. Examples of essential oils are thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract.

Examples of commercial products are Crina® (DSM Nutritional Products); Cinergy™, Cinergy™ FIT, Biacid™, (Cargill), Digesta®(R) and Dige®rom(R) DC (Biomin) and Envivo EO (DuPont Animal Nutrition).

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are propionic acid, formic acid, citric acid, lactic acid, sorbic acid, malic acid, acetic acid, fumaric acid, benzoic acid, butyric acid and tartaric acid or their salt (typically sodium or potassium salt such as potassium diformate or sodium butyrate).

Examples of commercial products are V®Vitall(R) (DSM Nutritional Produ®), Amas®R), Lupris®R), Lupr®rain(R),®pro-Cid(®Lupro-Mix(R), Lupro-Mix(R) NA (BASF), n-Butyric Acid AF (OXEA), Biacid™, Prohacid™ Classic and Prohacid™Avance™ (Cargill), Biotronic(R) (Biomin) and Adimix Precision (Nutriad).

Further, optional, feed-additive ingredients are colouring agents, e.g., carotenoids such as beta-carotene, astaxanthin, and lutein; aroma compounds; stabilisers; antimicrobial peptides; polyunsaturated fatty acids; reactive oxygen generating species; and/or at least one other enzyme selected from amongst another pectinase (EC 3.2.1.8); and/or beta-glucanase (EC 3.2.1.4 or EC 3.2.1.6).

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Usually fat- and water-soluble vitamins, as well as trace minerals form part of a so-called premix intended for addition to the feed, whereas macro minerals are usually separately added to the feed. A premix enriched with a fiber-degrading enzyme of the invention is an example of an animal feed additive of the invention.

The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the invention can be added to feed ingredients or to the drinking water as solids (for example as water soluble powder) or liquids.

In a particular embodiment, the animal feed additive of the invention is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

WO 01/58275 corresponds to U.S. Pat. No. 6,960,462 which is hereby incorporated by reference.

An animal feed composition according to the invention has a crude protein content of 50-800 g/kg (preferably 50-600 g/kg, more preferably 60-500 g/kg, even more preferably 70-500, and most preferably 80-400 g/kg) and furthermore comprises at least one fiber-degrading enzyme as claimed herein. In additional preferred embodiments, the crude protein content is 150-800, 160-800, 170-8-0, 180-800, 190-800, or 200-800—all in g/kg (dry matter). In particular embodiments, the crude protein content comes from oil seed material of the present invention.

Furthermore, or in the alternative (to the crude protein content indicated above), the animal feed composition suitably has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg. In an embodiment, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e., Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington DC).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen bv, Wageningen. ISBN 90-71463-12-5.

In a further aspect, the present invention relates to a method of improving the Average Metabolizable Energy of plant-based diet in a monogastric animal comprising administering an animal feed additive of the present invention or the animal feed of the present invention.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In particular embodiments, the animal feed composition of the invention contains 0-80% oil seed material.

Animal diets can, e.g., be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, a solid enzyme formulation is typically added before or during the mixing step; and a liquid enzyme preparation is typically added after the pelleting step. The enzyme may also be incorporated in a feed additive or premix, as described above.

In a preferred embodiment, the animal feed has been pelleted. The animal feed may be treated with the enzyme of the invention before the pelleting step or sprayed on after the pelleting step.

In a further aspect, the present invention relates to use of a combination of a polypeptide having rhamno-galacturonan lyase activity and a polypeptide having galactanase activity in an animal feed or animal feed additive.

In a further aspect, the present invention relates to an animal feed additive comprising a polypeptide having rhamno-galacturonan lyase activity and a polypeptide having galactanase activity; preferably, the present invention relates to an animal feed additive comprising a combination of rhamnogalacturonan lyase and endo-beta-1,4-galactanase; a combination of rhamnogalacturonan lyase and pectin lyase; a combination of endo-beta-1,4-galactanase and pectin lyase; or a combination of xyloglucan-specific endo-beta-1,4-glucanase/endo-xyloglucanase and pectin lyase.

In one embodiment, the polypeptide having rhamno-galacturonan lyase activity is RGL_1. In a further embodiment, the polypeptide having rhamno-galacturonan lyase activity is selected from the group consisting of:
(a) a rhamnogalacturonan lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 1 or a mature polypeptide of SEQ ID NO: 1; preferably a rhamnogalacturonan lyase obtained or obtainable from Aspergillus; more preferably, a rhamnogalacturonan lyase obtained or obtainable from Aspergillus aculeatus;
(b) a rhamnogalacturonan lyase derived from SEQ ID NO: 1 or a mature polypeptide of SEQ ID NO: 1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a rhamnogalacturonan lyase derived from (a), or (b), wherein the N- and/or C-terminal end has been extended by addition of one or more amino acids, e.g., 1 or 2 or 3 or 4 or 5 or 6 or 7 or 8 or 9 or 10 amino acids; and
(d) a fragment of the rhamnogalacturonan lyase of (a), (b), or (c), having the rhamnogalacturonan lyase activity.

In a further embodiment, the polypeptide having galactanase activity is a Glycoside hydrolase family (GH) 53 (GH53). In a further embodiment, the polypeptide having galactanase activity is selected from the group consisting of:
(a) a galactanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 5 or a mature polypeptide of SEQ ID NO: 5; preferably a galactanase obtained or obtainable from Cohnella sp; more preferably, a galactanase obtained or obtainable from Cohnella sp-60555;
(b) a galactanase derived from SEQ ID NO: 5 or a mature polypeptide of SEQ ID NO: 5 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a galactanase derived from (a) or (b), wherein the N- and/or C-terminal end has been extended by addition of one or more amino acids, e.g., 1 or 2 or 3 or 4 or 5 or 6 or 7 or 8 or 9 or 10 amino acids; and
(d) a fragment of the galactanase of (a), (b), or (c), having the galactanase activity.

In a further aspect, the present invention relates to an animal feed additive comprising a polypeptide having rhamno-galacturonan lyase activity and a polypeptide having galactanase activity. Apart from a polypeptide having rhamno-galacturonan lyase activity and a polypeptide having galactanase activity of the invention, the animal feed additives of the invention further comprise one or more additional components selected from the group consisting of: one or more vitamins; one or more minerals; one or more amino acids; one or more phytogenics; one or more prebiotics; one or more organic acids; and one or more other feed ingredients.

In a further aspect, the present invention relates to a polypeptide having xyloglucan-specific endo-1,4-beta-glucanase activity, selected from the group consisting of:
(a) a polypeptide having at least 99.7%, at least 99.8%, at least 99.9% or 100% sequence identity to SEQ ID NO: 3 or the mature polypeptide of SEQ ID NO: 3;
(b) a polypeptide derived from SEQ ID NO: 3 or a mature polypeptide of SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide having at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID NO: 4 or the mature polypeptide of SEQ ID NO: 4;
(d) a polypeptide derived from SEQ ID NO: 4 or a mature polypeptide of SEQ ID NO: 4 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(e) a polypeptide derived from the polypeptide of (a), (b), (c) or (d), wherein the N- and/or C-terminal end has been extended by addition of one or more amino acids, e.g., 1 or 2 or 3 or 4 or 5 or 6 or 7 or 8 or 9 or 10 amino acids; and
(f) a fragment of the polypeptide of (a), (b), (c), (d) or (e);
   wherein the polypeptide has xyloglucan-specific endo-1,4-beta-glucanase activity.

In one embodiment, the polypeptide of the present invention comprises, consists essentially of, or consists of SEQ ID NO: 3, a mature polypeptide of SEQ ID NO: 3, SEQ ID NO: 4, or a mature polypeptide of SEQ ID NO: 4.

In a further aspect, the present invention relates to a polypeptide having xylogalacturonase activity, selected from the group consisting of:
(a) a polypeptide having at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID NO: 6 or the mature polypeptide of SEQ ID NO: 6;
(b) a polypeptide derived from SEQ ID NO: 6 or a mature polypeptide of SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7 or the mature polypeptide of SEQ ID NO: 7;
(d) a polypeptide derived from SEQ ID NO: 7 or a mature polypeptide of SEQ ID NO: 7 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(e) a polypeptide derived from the polypeptide of (a), (b), (c) or (d), wherein the N- and/or C-terminal end has been extended by addition of one or more amino acids, e.g., 1 or 2 or 3 or 4 or 5 or 6 or 7 or 8 or 9 or 10 amino acids; and
(f) a fragment of the polypeptide of (a), (b), (c), (d) or (e);
wherein the polypeptide has xylogalacturonase activity.

In one embodiment, the polypeptide of the present invention comprises, consists essentially of, or consists of SEQ ID NO: 6, a mature polypeptide of SEQ ID NO: 6, SEQ ID NO: 7, or a mature polypeptide of SEQ ID NO: 7.

In further aspect, the present invention relates to a polypeptide having rhamnogalacturonan lyase activity, selected from the group consisting of:
(a) a polypeptide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 9 or the mature polypeptide of SEQ ID NO: 9;
(b) a polypeptide derived from SEQ ID NO: 9 or a mature polypeptide of SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide derived from the polypeptide of (a) or (b), wherein the N- and/or C-terminal end has been extended by addition of one or more amino acids, e.g., 1 or 2 or 3 or 4 or 5 or 6 or 7 or 8 or 9 or 10 amino acids; and
(d) a fragment of the polypeptide of (a), (b), or (c);
wherein the polypeptide has rhamnogalacturonan lyase activity.

In one embodiment, the polypeptide of the present invention comprises, consists essentially of, or consists of SEQ ID NO: 9, or a mature polypeptide of SEQ ID NO: 9.

In further aspect, the present invention relates to a polypeptide having xyloglucan-specific endo-b-1,4-glucanase/endo-xyloglucanase, selected from the group consisting of:
(a) a polypeptide having at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 10 or the mature polypeptide of SEQ ID NO: 10;
(b) a polypeptide derived from SEQ ID NO: 10 or a mature polypeptide of SEQ ID NO: 10 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide derived from the polypeptide of (a) or (b), wherein the N- and/or C-terminal end has been extended by addition of one or more amino acids, e.g., 1 or 2 or 3 or 4 or 5 or 6 or 7 or 8 or 9 or 10 amino acids; and
(d) a fragment of the polypeptide of (a), (b), or (c);
wherein the polypeptide has xyloglucan-specific endo-b-1,4-glucanase/endo-xyloglucanase activity.

In one embodiment, the polypeptide of the present invention comprises, consists essentially of, or consists of SEQ ID NO: 10, or a mature polypeptide of SEQ ID NO: 10.

### Polynucleotides

In a further aspect, the present invention relates to a polynucleotide encoding the polypeptide of the present invention.

The polynucleotide may be a genomic DNA, a cDNA, a synthetic DNA, a synthetic RNA, a mRNA, or a combination thereof. The polynucleotide may be cloned from a strain of Aspergillus or Penicillium or Cohnella, or a related organism and thus, for example, may be a polynucleotide sequence encoding a variant of the polypeptide of the invention.

In one embodiment the polynucleotide encoding the rhamnogalacturonan lyase of the present invention is isolated from an Aspergillus or Penicillium cell.

In one embodiment, the polynucleotide encoding the xyloglucan-specific endo-1,4-beta-glucanase of the present invention is isolated from an Aspergillus cell.

In one embodiment, the polynucleotide encoding the galactanase of the present invention is isolated from a Cohnella cell.

The polynucleotide may also be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

In one embodiment, the polynucleotide is isolated.

In another embodiment, the polynucleotide is purified.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. Techniques for modifying polynucleotides utilizing recombinant DNA methods are well known in the art.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Suitable leaders for bacterial host cells are described by Hambraeus et al., 2000, Microbiology 146(12): 3051-3059, and by Kaberdin and Blssi, 2006, FEMS Microbiol. Rev. 30(6): 967-979.

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. Any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17: 52.

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a polypeptide of the present invention.

A construct or vector comprising a polynucleotide is introduced into a host cell so that the construct or vector is maintained as a chromosomal integrant or as a self-replicating extrachromosomal vector as described earlier. The choice of a host cell will to a large extent depend upon the gene encoding the polypeptide and its source. The polypeptide can be native or heterologous to the recombinant host cell. Also, at least one of the one or more control sequences can be heterologous to the polynucleotide encoding the polypeptide. The recombinant host cell may comprise a single copy, or at least two copies, e.g., three, four, five, or more copies of the polynucleotide of the present invention.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70: 406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

In an aspect, the host cell is isolated.

In another aspect, the host cell is purified.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is an Aspergillus or Penicillium or Cohnella cell. In another aspect, the cell is an Aspergillus aculeatus, Aspergillus tubingensis, Aspergillus luchuensis, Penicillium oxalicum or Penicillium rubens cell. In another aspect, the cell is Cohnella sp-60555.

The present invention also relates to methods of producing a polypeptide of the present invention, comprising (a) cultivating a recombinant host cell of the present invention under conditions conducive for production of the polypeptide; and optionally, (b) recovering the polypeptide.

The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the polypeptide is not recovered.

In a further aspect, the present invention relates to an animal feed additive comprising the polypeptide of the present invention.

In a further aspect, the present invention relates to an animal feed, comprising the polypeptide of the present invention or the animal feed additive of the present invention.

The invention is further defined by the following numbered paragraphs:
1. A method of improving the nutritional value of an animal feed comprising an oil seed material, comprising adding a fiber-degrading enzyme to said animal feed.
2. A method of improving growth performance of an animal, comprising administrating to the animal a fiber-degrading enzyme, an animal feed or animal feed additive comprising a fiber-degrading enzyme; preferably the growth performance is the growth rate, the feed conversion ratio, and/or the body weight gain.
3. The method according to paragraph 1 or 2, wherein the oil seed material is selected from the group consisting of soy bean, rapeseed, sunflower, peas, lupin, tepary bean, scarlet runner bean, slimjim bean, lima bean, French bean, Broad bean (fava bean), chickpea, lentil, peanut, linseed, cottonseed, or the combination thereof; or wherein the oil seed material is processed; preferably the oil seed material is selected from the group consisting of soy bean meal, rapeseed meal, sunflower meal, peas meal, peanut meal, linseed meal, cottonseed meal, or the combination thereof.
4. The method according to any one of paragraphs 1 to 3, wherein the animal is a monogastric animal, preferably, the monogastric animal is selected from the group consisting of pigs or swine (including, but not limited to, piglets, growing pigs, and sows); poultry such as turkeys, ducks and chicken (including but not limited to broiler chicks, layers).
5. The method according to any one of paragraphs 1 to 4, wherein the fiber-degrading enzyme is selected from the group consisting of a pectinase, xyloglucan specific endo 1,4-beta glucanase, xyloglucan-specific endo-beta-1,4-glucanase/endo-xyloglucanase, and the combination thereof; preferably the fiber-degrading enzyme is a pectinase; more preferably the fiber-degrading enzyme is one or more pectinases selected from the group consisting of rhamnogalacturonan lyase, endo-beta-1,4-galactanase, polygalacturonase, rhamnogalacturonanase, pectin methyl esterase, pectin lyase, pectin acetyl esterase, galactan endo-beta-1,3-galactanase, xylogalacturonase, and the combination thereof; or wherein the fiber-degrading enzyme is selected from the group consisting of a pectinase, xyloglucan specific endo 1,4-beta glucanase, xyloglucan-specific endo-beta-1,4-glucanase/endo-xyloglucanase, and the combination thereof; preferably the fiber-degrading enzyme is a pectinase; more preferably the fiber-degrading enzyme is one or more pectinases selected from the group consisting of rhamnogalacturonan lyase (alpha-L-rhamnopyranosyl-1,4-alpha-D-galactopyranosyluronate endolyase, EC 4.2.2.23), endo-beta-1,4-galactanase (EC 3. 2. 1. 89), polygalacturonase (1,4-alpha-D-galacturonan glycanohydrolase, EC 3.2.1.15 and EC 3.2.1.67), rhamnogalacturonanase (rhamnogalacturonan alpha-D-galacturonic acid-1,2-alpha-L-rhamnose hydrolase, EC 3.2.1.171), pectin methyl esterase (pectin pectyl hydrolase, EC 3.1.1.11), pectin lyase ((1,4)-6-O-methyl-alpha-D-galacturonan lyase, EC 4.2.2.10), pectin acetyl esterase (acetic ester acetylhydrolase, EC 3.1.1.6), galactan endo-beta-1,3-galactanase (EC 3.2.1.181), xylogalacturonase, and the combination thereof.
6. The method according to any one of paragraphs 1-5, wherein the fiber-degrading enzyme is a pectinase selected from the group consisting of rhamnogalacturonan lyase, endo-beta-1,4-galactanase, pectin lyase and the combination thereof; preferably, the fiber-degrading enzyme is selected from the group consisting of a combination of rhamnogalacturonan lyase and endo-beta-1,4-galactanase; a combination of rhamnogalacturonan lyase and pectin lyase; a combination of endo-beta-1,4-galactanase and pectin lyase; and a combination of xyloglucan-specific endo-beta-1,4-glucanase/endo-xyloglucanase and pectin lyase.
7. The method according to any one of paragraphs 1 to 6 wherein the fiber-degrading enzyme is obtained from or obtainable from Aspergillus or Penicillium or Cohnella; preferably, the fiber-degrading enzyme is a rhamnogalacturonan lyase obtained from an Aspergillus or Penicillium, e.g., a rhamnogalacturonan lyase obtained from A. aculeatus or Penicillium oxalicum; the fiber-degrading enzyme is an endo-polygalacturonase obtained from Aspergillus, e.g., an endo-polygalacturonase obtained from A. aculeatus; the fiber-degrading enzyme is a xyloglucan-specific endo-1,4-beta-glucanase obtained from or obtainable from Aspergillus, e.g., a xyloglucan-specific endo-1,4-beta-glucanase obtained from A. aculeatus or A. luchuensis; the fiber-degrading enzyme is a galactanase obtained from or obtainable from Cohnella, e.g., a galactanase obtained from Cohnella sp-60555; the fiber-degrading enzyme is a xylogalacturonase obtained from or obtainable from Aspergillus, e.g., a xylogalacturonase obtained from Aspergillus tubingensis or Aspergillus aculeatus; the fiber-degrading enzyme is a pectin lyase obtained from or obtainable from Aspergillus, e.g., a pectin lyase obtained from Aspergillus aculeatus; the fiber-degrading enzyme is an endo-beta-1,4-glucanase/endo-xyloglucanase obtained from or obtainable from Penicillium, e.g., an endo-beta-1,4-glucanase/endo-xyloglucanase obtained from Penicillium rubens.
8. A method of generating a prebiotic in-situ in an oil seed based animal feed comprising adding a fiber-degrading enzyme to said animal feed.
9. A method of decreasing an insoluble pectin fraction in an oil seed based animal feed comprising adding a fiber-degrading enzyme to said animal feed.
10. A method of improving intestinal health of a monogastric animal comprising administrating an oil seed based animal feed to said animal wherein said animal feed comprises a fiber-degrading enzyme.
11. The method according to paragraph 10, wherein the fiber-degrading enzyme degrades the pectin polysaccharides of said oil seed material so as to generate prebiotic oligomers and polymers comprising pectin oligosaccharides.
12. The method according to paragraph 11, wherein the cecal butyrate levels in situ in said animal is increased.
13. The method according to paragraph 11, wherein the microbiota composition in said animal is altered.
14. A method for in-situ production of prebiotics in monogastric animals comprising administrating an enzyme-enriched oil seed based animal feed to said animal wherein said animal feed comprises a fiber-degrading enzyme.
15. A method of causing a butyrogenic effect in a monogastric animal comprising administrating an oil seed based animal feed to said animal wherein said animal feed comprises a fiber-degrading enzyme.
16. The method according to any one of paragraphs 8 to 15, wherein the oil seed material is selected from the group consisting of soy bean, rapeseed, sunflower, peas, lupin, tepary bean, scarlet runner bean, slimjim bean, lima bean, French bean, Broad bean (fava bean), chickpea, lentil, peanut, linseed, cottonseed, or the combination thereof; or wherein the oil seed material is processed, preferably the oil seed material is selected from the group consisting of soy bean meal, rapeseed meal, sunflower meal, peas meal, peanut meal, linseed meal, cottonseed meal, or the combination thereof.
17. The method according to any one of paragraphs 8 to 16, wherein the animal is a monogastric animal, preferably, the monogastric animal is selected from the group consisting of pigs or swine (including, but not limited to, piglets, growing pigs, and sows); poultry such as turkeys, ducks and chicken (including but not limited to broiler chicks, layers).
18. The method according to any one of paragraphs 8 to 17, wherein the fiber-degrading enzyme is selected from the group consisting of a pectinase, xyloglucan specific endo 1,4-beta glucanase, xyloglucan-specific endo-beta-1,4-glucanase/endo-xyloglucanase, and the combination thereof; preferably the fiber-degrading enzyme is a pectinase; more preferably the fiber-degrading enzyme is one or more pectinases selected from the group consisting of rhamnogalacturonan lyase, endo-beta-1,4-galactanase, polygalacturonase, rhamnogalacturonanase, pectin methyl esterase, pectin lyase, pectin acetyl esterase, galactan endo-beta-1,3-galactanase, xylogalacturonase, and the combination thereof.
19. The method according to any one of paragraphs 8 to 18, wherein the fiber-degrading enzyme is a pectinase selected from the group consisting of rhamnogalacturonan lyase, endo-beta-1,4-galactanase, pectin lyase and the combination thereof; preferably, the fiber-degrading enzyme is selected from the group consisting of a combination of rhamnogalacturonan lyase and endo-beta-1,4-galactanase; a combination of rhamnogalacturonan lyase and pectin lyase; a combination of endo-beta-1,4-galactanase and pectin lyase; and a combination of xyloglucan-specific endo-beta-1,4-glucanase/endo-xyloglucanase and pectin lyase.
20. The method according to any one of paragraphs 8 to 19, wherein the fiber-degrading enzyme is obtained from or obtainable from Aspergillus or Penicillium or Cohnella; preferably, the fiber-degrading enzyme is a rhamnogalacturonan lyase obtained from an Aspergillus or Penicillium, e.g., a rhamnogalacturonan lyase obtained from A. aculeatus or Penicillium oxalicum; the fiber-degrading enzyme is an endo-polygalacturonase obtained from Aspergillus, e.g., an endo-polygalacturonase obtained from A. aculeatus; the fiber-degrading enzyme is a xyloglucan-specific endo-1,4-beta-glucanase obtained from or obtainable from Aspergillus, e.g., a xyloglucan-specific endo-1,4-beta-glucanase obtained from A. aculeatus or A. luchuensis; the fiber-degrading enzyme is a galactanase obtained from or obtainable from Cohnella, e.g., a galactanase obtained from Cohnella sp-60555; the fiber-degrading enzyme is a xylogalacturonase obtained from or obtainable from Aspergillus, e.g., a xylogalacturonase obtained from Aspergillus tubingensis or Aspergillus aculeatus; the fiber-degrading enzyme is a pectin lyase obtained from or obtainable from Aspergillus, e.g., a pectin lyase obtained from Aspergillus aculeatus; the fiber-degrading enzyme is an endo-beta-1,4-glucanase/endo-xyloglucanase obtained from or obtainable from Penicillium, e.g., an endo-beta-1,4-glucanase/endo-xyloglucanase obtained from Penicillium rubens.
21. An animal feed comprising a fiber-degrading enzyme and an oil seed material wherein the feed comprises oil seed material in an amount of 10 to 500 g/kg feed and the fiber-degrading enzyme in an amount of 0.1 to 500 mg enzyme protein/kg of feed.
22. Use of a fiber-degrading enzyme in preparation of an enzyme-enriched animal feed, wherein said animal feed is an oil seed based animal feed.
23. The animal feed according to paragraph 21, the use according to paragraph 22, wherein the oil seed material is selected from the group consisting of soy bean, rapeseed, sunflower, peas, peanut, linseed, cottonseed, or the combination thereof; preferably the oil seed material is selected from the group consisting of soy bean meal, rapeseed meal, sunflower meal, peas meal, peanut meal, linseed meal, cottonseed meal, or the combination thereof.
24. An animal feed additive, comprising a fiber-degrading enzyme and one or more additional components selected from the group consisting of: one or more vitamins; one or more minerals; one or more amino acids; one or more phytogenics; one or more prebiotics; one or more organic acids; and one or more other feed ingredients.
25. The animal feed according to paragraph 21, the use according to paragraph 22, the animal feed additive according to paragraph 24, wherein the animal is a monogastric animal, preferably, the monogastric animal is selected from the group consisting of pigs or swine (including, but not limited to, piglets, growing pigs, and sows); poultry such as turkeys, ducks and chicken (including but not limited to broiler chicks, layers).
26. The animal feed according to paragraph 21, the use according to paragraph 22, the animal feed additive according to paragraph 24, wherein the fiber-degrading enzyme is selected from the group consisting of a pectinase, xyloglucan specific endo 1,4-beta glucanase, xyloglucan-specific endo-beta-1,4-glucanase/endo-xyloglucanase, and the combination thereof; preferably the fiber-degrading enzyme is a pectinase; more preferably the fiber-degrading enzyme is one or more pectinases selected from the group consisting of rhamnogalacturonan lyase (alpha-L-rhamnopyranosyl-1,4-alpha-D-galactopyranosyluronate endolyase, EC 4.2.2.23), endo-beta-1,4-galactanase (EC 3. 2. 1. 89), polygalacturonase (1,4-alpha-D-galacturonan glycanohydrolase, EC 3.2.1.15 and EC 3.2.1.67), rhamnogalacturonanase (rhamnogalacturonan alpha-D-galacturonic acid-1,2-alpha-L-rhamnose hydrolase, EC 3.2.1.171), pectin methyl esterase (pectin pectyl hydrolase, EC 3.1.1.11), pectin lyase ((1,4)-6-O-methyl-alpha-D-galacturonan lyase, EC 4.2.2.10), pectin acetyl esterase (acetic ester acetylhydrolase, EC 3.1.1.6), galactan endo-beta-1,3-galactanase (EC 3.2.1.181), xylogalacturonase, and the combination thereof.
27. The animal feed according to paragraph 21, the use according to paragraph 22, the animal feed additive according to paragraph 24, wherein the fiber-degrading enzyme is a pectinase; preferably the fiber-degrading enzyme is a pectinase selected from the group consisting of rhamnogalacturonan lyase, endo-beta-1,4-galactanase, and the combination thereof.
28. The animal feed according to paragraph 21, the use according to paragraph 22, the animal feed additive according to paragraph 24, wherein the fiber-degrading enzyme is obtained from or obtainable from Aspergillus or Penicillium or Cohnella; preferably, the fiber-degrading enzyme is a rhamnogalacturonan lyase obtained from an Aspergillus or Penicillium, e.g., a rhamnogalacturonan lyase obtained from A. aculeatus or Penicillium oxalicum; the fiber-degrading enzyme is an endo-polygalacturonase obtained from Aspergillus, e.g., an endo-polygalacturonase obtained from A. aculeatus; the fiber-degrading enzyme is a xyloglucan-specific endo-1,4-beta-glucanase obtained from or obtainable from Aspergillus, e.g., a xyloglucan-specific endo-1,4-beta-glucanase obtained from A. aculeatus or A. luchuensis; the fiber-degrading enzyme is a galactanase obtained from or obtainable from Cohnella, e.g., a galactanase obtained from Cohnella sp-60555; the fiber-degrading enzyme is a xylogalacturonase obtained from or obtainable from Aspergillus, e.g., a xylogalacturonase obtained from Aspergillus tubingensis or Aspergillus aculeatus; the fiber-degrading enzyme is a pectin lyase obtained from or obtainable from Aspergillus, e.g., a pectin lyase obtained from Aspergillus aculeatus; the fiber-degrading enzyme is an endo-beta-1,4-glucanase/endo-xyloglucanase obtained from or obtainable from Penicillium, e.g., an endo-beta-1,4-glucanase/endo-xyloglucanase obtained from Penicillium rubens.
29. The animal feed according to paragraph 21, the use according to paragraph 22, the animal feed additive according to paragraph 24, wherein the fiber-degrading enzyme is selected from the group consisting of:
   (a) a rhamnogalacturonan lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 1 or a mature polypeptide of SEQ ID NO: 1;
   (b) a rhamnogalacturonan lyase derived from SEQ ID NO: 1 or a mature polypeptide of SEQ ID NO: 1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a rhamnogalacturonan lyase derived from (a), or (b), wherein the N- and/or C-terminal end has been extended by addition of one or more amino acids, e.g., 1 or 2 or 3 or 4 or 5 or 6 or 7 or 8 or 9 or 10 amino acids;
   (d) a fragment of the rhamnogalacturonan lyase of (a), (b), or (c), having the rhamnogalacturonan lyase activity;
   (e) an endo-polygalacturonase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 2 or a mature polypeptide of SEQ ID NO: 2;
   (f) an endo-polygalacturonase derived from SEQ ID NO: 2 or a mature polypeptide of SEQ ID NO: 2 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (g) an endo-polygalacturonase derived from (e), or (f), wherein the N- and/or C-terminal end has been extended by addition of one or more amino acids, e.g., 1 or 2 or 3 or 4 or 5 or 6 or 7 or 8 or 9 or 10 amino acids;
   (h) a fragment of the endo-polygalacturonase of (e), (f), or (g), having the endo-polygalacturonase activity;
   (i) a xyloglucan-specific endo-1,4-beta-glucanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 3 or a mature polypeptide of SEQ ID NO: 3;
   (j) a xyloglucan-specific endo-1,4-beta-glucanase derived from SEQ ID NO: 3 or a mature polypeptide of SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (k) a xyloglucan-specific endo-1,4-beta-glucanase derived from (i), or (j), wherein the N- and/or C-terminal end has been extended by addition of one or more amino acids, e.g., 1 or 2 or 3 or 4 or 5 or 6 or 7 or 8 or 9 or 10 amino acids;
   (l) a fragment of the xyloglucan-specific endo-1,4-beta-glucanase of (i), (j), or (k), having the xyloglucan-specific endo-1,4-beta-glucanase activity;
   (m) a xyloglucan-specific endo-1,4-beta-glucanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 4 or a mature polypeptide of SEQ ID NO: 4;
   (n) a xyloglucan-specific endo-1,4-beta-glucanase derived from SEQ ID NO: 4 or a mature polypeptide of SEQ ID NO: 4 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (o) a xyloglucan-specific endo-1,4-beta-glucanase derived from (m), or (n), wherein the N- and/or C-terminal end has been extended by addition of one or more amino acids, e.g., 1 or 2 or 3 or 4 or 5 or 6 or 7 or 8 or 9 or 10 amino acids; and
   (p) a fragment of the xyloglucan-specific endo-1,4-beta-glucanase of (m), (n), or (o), having a xyloglucan-specific endo-1,4-beta-glucanase activity;
   (q) a galactanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 5 or a mature polypeptide of SEQ ID NO: 5;
   (r) a galactanase derived from SEQ ID NO: 5 or a mature polypeptide of SEQ ID NO: 5 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (s) a galactanase derived from (q) or (r), wherein the N- and/or C-terminal end has been extended by addition of one or more amino acids, e.g., 1 or 2 or 3 or 4 or 5 or 6 or 7 or 8 or 9 or 10 amino acids;
   (t) a fragment of the galactanase of (q), (r), or (s), having the galactanase activity;
   (u) a xylogalacturonase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 6 or a mature polypeptide of SEQ ID NO: 6;
   (v) a xylogalacturonase derived from SEQ ID NO: 6 or a mature polypeptide of SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (w) a xylogalacturonase derived from (u) or (v), wherein the N- and/or C-terminal end has been extended by addition of one or more amino acids, e.g., 1 or 2 or 3 or 4 or 5 or 6 or 7 or 8 or 9 or 10 amino acids;
   (x) a fragment of the xylogalacturonase of (u), (v), or (w), having the xylogalacturonase activity;
   (y) a xylogalacturonase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 7 or a mature polypeptide of SEQ ID NO: 7;
   (z) a xylogalacturonase derived from SEQ ID NO: 7 or a mature polypeptide of SEQ ID NO: 7 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (aa) a xylogalacturonase derived from (y) or (z), wherein the N- and/or C-terminal end has been extended by addition of one or more amino acids, e.g., 1 or 2 or 3 or 4 or 5 or 6 or 7 or 8 or 9 or 10 amino acids;
   (bb) a fragment of the xylogalacturonase of (y), (z) or (aa) having the xylogalacturonase activity;
   (cc) a pectin lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 8 or a mature polypeptide of SEQ ID NO: 8;
   (dd) a pectin lyase derived from SEQ ID NO: 8 or a mature polypeptide of SEQ ID NO: 8 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (ee) a pectin lyase derived from (cc) or (dd), wherein the N- and/or C-terminal end has been extended by addition of one or more amino acids, e.g., 1 or 2 or 3 or 4 or 5 or 6 or 7 or 8 or 9 or 10 amino acids;
   (ff) a fragment of the pectin lyase of (cc), (dd), or (ee) having the pectin lyase activity;
   (gg) a rhamnogalacturonan lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 9 or a mature polypeptide of SEQ ID NO: 9;
   (hh) a rhamnogalacturonan lyase derived from SEQ ID NO: 9 or a mature polypeptide of SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (ii) a rhamnogalacturonan lyase derived from (gg) or (hh), wherein the N- and/or C-terminal end has been extended by addition of one or more amino acids, e.g., 1 or 2 or 3 or 4 or 5 or 6 or 7 or 8 or 9 or 10 amino acids; and
   (jj) a fragment of the rhamnogalacturonan lyase of (gg), (hh) or (ii) having rhamnogalacturonan lyase activity;
   (kk) a xyloglucan-specific endo-b-1,4-glucanase/endo-xyloglucanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 10 or a mature polypeptide of SEQ ID NO: 10;
   (ll) a xyloglucan-specific endo-b-1,4-glucanase/endo-xyloglucanase derived from SEQ ID NO: 10 or a mature polypeptide of SEQ ID NO: 10 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (mm) a xyloglucan-specific endo-b-1,4-glucanase/endo-xyloglucanase derived from (kk) or (ll), wherein the N- and/or C-terminal end has been extended by addition of one or more amino acids, e.g., 1 or 2 or 3 or 4 or 5 or 6 or 7 or 8 or 9 or 10 amino acids; and
   (nn) a fragment of the xyloglucan-specific endo-b-1,4-glucanase/endo-xyloglucanase of (kk), (ll) or (mm) having a xyloglucan-specific endo-b-1,4-glucanase/endo-xyloglucanase activity.
30. Use of a combination of a polypeptide having rhamno-galacturonan lyase activity and a polypeptide having galactanase activity in an animal feed or animal feed additive; use of a combination of rhamnogalacturonan lyase and endo-beta-1,4-galactanase in an animal feed or animal feed additive; use of a combination of rhamnogalacturonan lyase and pectin lyase in an animal feed or animal feed additive; use of a combination of endo-beta-1,4-galactanase and pectin lyase in an animal feed or animal feed additive; or use of a combination of xyloglucan-specific endo-beta-1,4-glucanase/endo-xyloglucanase and pectin lyase in an animal feed or animal feed additive.
31. The use according to paragraph 30 wherein the polypeptide having rhamno-galacturonan lyase activity is RGL_1.
32. The use according to paragraph 31, wherein the polypeptide having rhamno-galacturonan lyase activity is selected from the group consisting of
   (a) a rhamnogalacturonan lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 1 or a mature polypeptide of SEQ ID NO: 1; preferably a rhamnogalacturonan lyase obtained or obtainable from Aspergillus; more preferably, a rhamnogalacturonan lyase obtained or obtainable from Aspergillus aculeatus;
   (b) a rhamnogalacturonan lyase derived from SEQ ID NO: 1 or a mature polypeptide of SEQ ID NO: 1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a rhamnogalacturonan lyase derived from (a), or (b), wherein the N- and/or C-terminal end has been extended by addition of one or more amino acids, e.g., 1 or 2 or 3 or 4 or 5 or 6 or 7 or 8 or 9 or 10 amino acids; and
   (d) a fragment of the rhamnogalacturonan lyase of (a), (b), or (c), having the rhamnogalacturonan lyase activity.
33. The use according to paragraph 30 wherein the polypeptide having galactanase activity is a GH53.
34. The use according to paragraph 30, wherein the polypeptide having galactanase activity is selected from the group consisting of:
   (a) a galactanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 5 or a mature polypeptide of SEQ ID NO: 5; preferably a galactanase obtained or obtainable from Cohnella sp; more preferably, a galactanase obtained or obtainable from Cohnella sp-60555;
   (b) a galactanase derived from SEQ ID NO: 5 or a mature polypeptide of SEQ ID NO: 5 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a galactanase derived from (a) or (b), wherein the N- and/or C-terminal end has been extended by addition of one or more amino acids, e.g., 1 or 2 or 3 or 4 or 5 or 6 or 7 or 8 or 9 or 10 amino acids; and
   (d) a fragment of the galactanase of (a), (b), or (c), having the galactanase activity.
35. An animal feed additive comprising a polypeptide having rhamno-galacturonan lyase activity and a polypeptide having galactanase activity; preferably an animal feed additive comprising a combination of rhamnogalacturonan lyase and endo-beta-1,4-galactanase; a combination of rhamnogalacturonan lyase and pectin lyase; a combination of endo-beta-1,4-galactanase and pectin lyase; or a combination of xyloglucan-specific endo-beta-1,4-glucanase/endo-xyloglucanase and pectin lyase.
36. The animal feed additive according to paragraph 35 wherein the polypeptide having rhamno-galacturonan lyase activity is RGL_1.
37. The animal feed additive according to paragraph 35 or 36, wherein the polypeptide having rhamno-galacturonan lyase activity is selected from the group consisting of
   (a) a rhamnogalacturonan lyase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 1 or a mature polypeptide of SEQ ID NO: 1; preferably a rhamnogalacturonan lyase obtained or obtainable from Aspergillus; more preferably, a rhamnogalacturonan lyase obtained or obtainable from Aspergillus aculeatus;
   (b) a rhamnogalacturonan lyase derived from SEQ ID NO: 1 or a mature polypeptide of SEQ ID NO: 1 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a rhamnogalacturonan lyase derived from (a), or (b), wherein the N- and/or C-terminal end has been extended by addition of one or more amino acids, e.g., 1 or 2 or 3 or 4 or 5 or 6 or 7 or 8 or 9 or 10 amino acids; and
   (d) a fragment of the rhamnogalacturonan lyase of (a), (b), or (c), having the rhamnogalacturonan lyase activity.
38. The animal feed additive according to paragraph 35 wherein the polypeptide having galactanase activity is a GH53.
39. The animal feed additive according to paragraph 35 or 38, wherein the polypeptide having galactanase activity is selected from the group consisting of:
   (a) a galactanase having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 5 or a mature polypeptide of SEQ ID NO: 5; preferably a galactanase obtained or obtainable from Cohnella sp; more preferably, a galactanase obtained or obtainable from Cohnella sp-60555;
   (b) a galactanase derived from SEQ ID NO: 5 or a mature polypeptide of SEQ ID NO: 5 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a galactanase derived from (a) or (b), wherein the N- and/or C-terminal end has been extended by addition of one or more amino acids, e.g., 1 or 2 or 3 or 4 or 5 or 6 or 7 or 8 or 9 or 10 amino acids; and
   (d) a fragment of the galactanase of (a), (b), or (c), having the galactanase activity.
40. The animal feed additive according to any one of paragraphs 35 to 39, further comprising one or more additional components selected from the group consisting of: one or more vitamins; one or more minerals; one or more amino acids; one or more phytogenics; one or more prebiotics; one or more organic acids; and one or more other feed ingredients.
41. An animal feed, comprising the animal feed additive according to any one of paragraphs 35-40, and an oil seed material.
42. A method of improving the Average Metabolizable Energy of plant-based diet in a monogastric animal comprising administering an animal feed additive as defined in any one of paragraphs 24, 35 to 40 or the animal feed according to any one of paragraphs 21 to 28 or 41.
43. A polypeptide having xyloglucan-specific endo-1,4-beta-glucanase activity, selected from the group consisting of:
   (a) a polypeptide having at least 99.7%, at least 99.8%, at least 99.9% or 100% sequence identity to SEQ ID NO: 3 or the mature polypeptide of SEQ ID NO: 3;
   (b) a polypeptide derived from SEQ ID NO: 3 or a mature polypeptide of SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a polypeptide having at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID NO: 4 or the mature polypeptide of SEQ ID NO: 4;
   (d) a polypeptide derived from SEQ ID NO: 4 or a mature polypeptide of SEQ ID NO: 4 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (e) a polypeptide derived from the polypeptide of (a), (b), (c) or (d), wherein the N- and/or C-terminal end has been extended by addition of one or more amino acids, e.g., 1 or 2 or 3 or 4 or 5 or 6 or 7 or 8 or 9 or 10 amino acids; and
   (f) a fragment of the polypeptide of (a), (b), (c), (d) or (e);

wherein the polypeptide has xyloglucan-specific endo-1,4-beta-glucanase activity.
44. The polypeptide of paragraph 43, comprising, consisting essentially of, or consisting of SEQ ID NO: 3, a mature polypeptide of SEQ ID NO: 3, SEQ ID NO: 4, or a mature polypeptide of SEQ ID NO: 4.
45. A polypeptide having xylogalacturonase activity, selected from the group consisting of:
   (a) a polypeptide having at least 99%, at least 99.1%, at least 99.2%, at least 99.3%, at least 99.4%, at least 99.5%, at least 99.6%, at least 99.7%, at least 99.8%, at least 99.9% or 100% sequence identity to SEQ ID NO: 6 or the mature polypeptide of SEQ ID NO: 6;
   (b) a polypeptide derived from SEQ ID NO: 6 or a mature polypeptide of SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a polypeptide having at least 82%, at least 83%, at least 84%, at least 85%, at least 86%, at least 87%, at least 88%, at least 89%, at least 90%, at least 91%, 92%, at least 93%, at least 94%, at least 95%, at least 96%, at least 97%, at least 98%, at least 99%, or 100% sequence identity to SEQ ID NO: 7 or the mature polypeptide of SEQ ID NO: 7;
   (d) a polypeptide derived from SEQ ID NO: 7 or a mature polypeptide of SEQ ID NO: 7 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (e) a polypeptide derived from the polypeptide of (a), (b), (c) or (d), wherein the N- and/or C-terminal end has been extended by addition of one or more amino acids, e.g., 1 or 2 or 3 or 4 or 5 or 6 or 7 or 8 or 9 or 10 amino acids; and
   (f) a fragment of the polypeptide of (a), (b), (c), (d) or (e);

wherein the polypeptide has xylogalacturonase activity.
46. The polypeptide of paragraph 45, comprising, consisting essentially of, or consisting of SEQ ID NO: 6, a mature polypeptide of SEQ ID NO: 6, SEQ ID NO: 7, or a mature polypeptide of SEQ ID NO: 7.
47. A polypeptide having rhamnogalacturonan lyase activity, selected from the group consisting of:
   (a) a polypeptide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 9 or the mature polypeptide of SEQ ID NO: 9;
   (b) a polypeptide derived from SEQ ID NO: 9 or a mature polypeptide of SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a polypeptide derived from the polypeptide of (a) or (b), wherein the N- and/or C-terminal end has been extended by addition of one or more amino acids, e.g., 1 or 2 or 3 or 4 or 5 or 6 or 7 or 8 or 9 or 10 amino acids; and
   (d) a fragment of the polypeptide of (a), (b), or (c);

wherein the polypeptide has rhamnogalacturonan lyase activity.
48. The polypeptide of paragraph 47, comprising, consisting essentially of, or consisting of SEQ ID NO: 9, or a mature polypeptide of SEQ ID NO: 9.
49. A polypeptide having xyloglucan-specific endo-b-1,4-glucanase/endo-xyloglucanase, selected from the group consisting of:
   (a) a polypeptide having at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 10 or the mature polypeptide of SEQ ID NO: 10;
   (b) a polypeptide derived from SEQ ID NO: 10 or a mature polypeptide of SEQ ID NO: 10 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a polypeptide derived from the polypeptide of (a) or (b), wherein the N- and/or C-terminal end has been extended by addition of one or more amino acids, e.g., 1 or 2 or 3 or 4 or 5 or 6 or 7 or 8 or 9 or 10 amino acids; and
   (d) a fragment of the polypeptide of (a), (b), or (c);

wherein the polypeptide has xyloglucan-specific endo-b-1,4-glucanase/endo-xyloglucanase activity.
50. The polypeptide of paragraph 49, comprising, consisting essentially of, or consisting of SEQ ID NO: 10, or a mature polypeptide of SEQ ID NO: 10.
51. A polynucleotide encoding the polypeptide of any one of paragraphs 43-50.
52. A nucleic acid construct or expression vector comprising the polynucleotide of paragraph 51, operably linked to one or more control sequences that direct the production of the polypeptide in an expression host.
53. A recombinant host cell comprising the nucleic acid construct or expression vector of paragraph 52.
54. An animal feed additive comprising the polypeptide of any one of paragraphs 43-50.
55. An animal feed, comprising the polypeptide of any one of paragraphs 43-50 or the animal feed additive of paragraph 54.

The present invention is further described by the following examples which should not be construed as limiting the scope of the invention.

### EXAMPLES

Chemicals used as buffers and substrates were commercial products of at least reagent grade.

### Enzymes Used in the Examples

| Enzyme | Family | Donor | Sequence | |
|---|---|---|---|---|
| rhamnogalacturonan | RGL_1 | Aspergillus | 100% sequence | Mature |
| lyase (Examples 1, 2, 8, | | aculeatus | identity with SEQ ID | polypeptide of |
| 9, 10) | | | NO: 826 of | SEQ ID NO: 1 |
| | | | WO2014081884-A1 | herein |
| endo-polygalacturonase | GH28_9 | Aspergillus | 100% sequence | Mature |
| (Example 4) | | aculeatus | identity with SEQ ID | polypeptide of |
| | | | NO: 692 of | SEQ ID NO: 2 |
| | | | WO2014081884-A1 | herein |
| endo-1,4-beta- | GH12_1 | Aspergillus | — | Mature |
| glucanase, xyloglucan- | | aculeatus | | polypeptide of |
| specific (Example 3) | | | | SEQ ID NO: 3 |
| | | | | herein |
| endo-1,4-beta- | GH12_1 | Aspergillus | — | Mature |
| glucanase, xyloglucan- | | luchuensis | | polypeptide of |
| specific (Example 3) | | | | SEQ ID NO: 4 |
| | | | | herein |
| galactanase (Examples | GH53 | Cohnella sp- | 100% sequence | Mature |
| 1, 2, 6, 7, 8, 9, 10) | | 60555 | identity with SEQ ID | polypeptide of |
| | | | NO: 2 of | SEQ ID NO: 5 |
| | | | WO2017202946-A1 | herein |
| xylogalacturonase | GH28_XGH | Aspergillus | — | Mature |
| (Example 5) | | tubingensis | | polypeptide of |
| | | | | SEQ ID NO: 6 |
| | | | | herein |
| xylogalacturonase | GH28_XGH | Aspergillus | — | Mature |
| (Example 5) | | aculeatus | | polypeptide of |
| | | | | SEQ ID NO: 7 |
| | | | | herein |
| pectin lyase (Example 8, | LYA1_4 | Aspergillus | 100% sequence | Mature |
| 9) | | aculeatus | identity with SEQ ID | polypeptide of |
| | | | NO: 10 in | SEQ ID NO: 8 |
| | | | WO2020002574-A1 | herein |
| rhamnogalacturonan | RGL_1 | Penicillium | — | Mature |
| lyase (Example 6, 7, 8, | | oxalicum | | polypeptide of |
| 9) | | | | SEQ ID NO: 9 |
| | | | | herein |
| putative endo-b-1,4- | GH5_4 | Penicillium | — | Mature |
| glucanase, endo-b-1,4- | | rubens | | polypeptide of |
| xylanase, endo-b-1,3- | | | | SEQ ID NO: 10 |
| 1,4-glucanase, | | | | herein |
| xyloglucan-specific | | | | |
| endo-b-1,4- | | | | |
| glucanase/endo- | | | | |
| xyloglucanase | | | | |
| (Example 8) | | | | |

### Example 1: Effect of a Rhamnogalacturonan Endolyase, an Endo-β-1,4-Galactanase and the Combination on Soybean Meal

The effect of a rhamnogalacturonan endolyase (RGL_1, A. aculeatus) and an endo-β-1,4-galactanase (GH53) for application in the animal feed on deproteinized soybean meal (SBM) was investigated in an in vitro experiment.

SBM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase® the enzymes were inactivated at 80 °C for 15 min. After centrifugation at 3,000 rpm at 0 °C for 15 min, the ethanol from the pellets was evaporated overnight in fume hood and the samples were freeze-dried.

Deproteinized SBM (3% dry matter) was incubated with a rhamnogalacturonan endolyase (RGL_1), an endo-β-1,4-galactanase (GH53), or a combination thereof in acetate buffer at pH 5.0 for 4 hours at 40 °C. Experiments were run in triplicates.

Following incubations with the enzymes, the solids were removed by centrifugation for 15 min at 4,700 g and 0 °C and 1,000 μL of the supernatant was subjected to acid hydrolysis (0.5 M H₂SO₄) for 50 min at 124 °C in the autoclave. The hydrolysate was filtered through 0.2 μm wwPTFE membrane (AcroPrep Advance 8582, PALL) and analyzed for its monosaccharide contents by high performance anion exchange chromatography combined with pulsed amperometric detection (HPAEC-PAD).

Monosaccharide separation was achieved on a CarboPac analytical PA1 column (4 mm×250 mm) with a CarboPac PA1 guard column (4 mm×50 mm) (Thermofisher) at a temperature of 30 °C with an eluent flow rate fixed at 1 mL/min. The elution of neutral monosaccharides was achieved with 10 mM NaOH as eluent within 14 min. In the sequence, the eluent concentration was increased to 500 mM and the acid monosaccharides were resolved within 7.5 min. The column was re-equilibrated with NaOH 10 mM for 10.5 min before injection of subsequent sample. The concentration of monosaccharides was calculated based on the standard curves of fucose, arabinose, rhamnose, galactose, glucose, xylose, mannose, galacturonic acid and glucuronic acid.

As shown in table 1, the RG-I lyase alone can solubilize rhamnose, arabinose, galactose and gal acid. The galactanase alone can solubilize arabinose and galactose. So the two enzymes are efficient in solubilizing pectin polymers on their own. When two enzymes are mixed, the effect is synergistic. As demonstrated by the experiments, the breakdown of rhamnose-, arabinose- and galacturonic acid-containing polysaccharides from SBM is increased by approximately 3-fold when the RG-I lyase is combined with the GH53 compared to the sum of the effect of the two enzymes separately, illustrating the synergy between the two enzyme products.

Table 1. Mean value of the amount of rhamnose, arabinose, galactose and galacturonic acid after treatment with no addition of enzyme (Control), with the addition of 20 ppm of RG-1 lyase (RGL_1), with the addition of 20 ppm of galactanase (GH53), and with a combination of the two enzymes at 20 ppm each. The values are given in % of the monosaccharide in soybean meal (SBM).

| | | | | Galacturonic |
|---|---|---|---|---|
| Treatment | Rhamnose | Arabinose | Galactose | acid |
| Control | 0.055 | 0.852 | 2.696 | 0.136 |
| RG-I lyase | 0.148 | 0.917 | 2.885 | 0.172 |
| Galactanase | 0.053 | 1.218 | 10.047 | 0.117 |
| RG-I lyase | 0.312 | 2.283 | 11.148 | 0.253 |
| and Galactanase | | | | |

### Example 2: The Effect of a Rhamnogalacturonan Endolyase, an Endo-β-1,4-Galactanase and the Combination on the Accumulation of Butyrate in In Vitro Fermentation of Rapeseed Meal by Chicken Caecal Microbiota

The effect of a rhamnogalacturonan endolyase (RGL_1, A. aculeatus) and an endo-β-1,4-galactanase (GH53) for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase®, the enzymes were inactivated at 80 °C for 15 min. After centrifugation at 3,000 rpm at 0 °C for 15 min, the ethanol from the pellet was evaporated overnight in fume hood and the samples were freeze-dried.

Deproteinized RSM (3% dry matter) with and without rhamnogalacturonan endolyase (20 ppm) and/or endo-β-1,4-galactanase (20 ppm) were diluted in anoxic sterile YCFA medium prepared as described by Duncan et al (Duncan, S. H., Hold, G. L., Barcenilla, A., Stewart, C. S. & Flint, H. J. Roseburia intestinalis sp. nov., a novel saccharolytic, butyrate-producing bacterium from human faeces. Int. J. Syst. Evol. Microbiol. 52, 1615-1620 (2002)) modified so the carbon source is 1% of non-starch polysaccharides from deproteinized RSM and the pH was adjusted to 6.5. Chicken caecal content from 35-day-old broiler was added to a final 1,000-fold dilution for the first round of fermentation. After 12 hours of fermentation at 37 °C, the first fermentation was used as inoculum for the second fermentation and so on. Fermentation supernatants were sampled after 12 hours and stored at −20 °C until analysis. Fermentations were run in five replicates.

The quantification of acetate, propionic acid and butyrate in the supernatants was achieved by running the samples in a high-performance liquid chromatography set-up equipped with an ion-exchange BioRad HPX-87H column with a BioRad Cation H precolumn at 60 °C and a refractive index detector.

The experiment demonstrates that the treatment with rhamnogalacturonan endolyase and with the galactanase separately increased the accumulation of butyrate by chicken caecal microbiota fermentation of RSM from 8.9 to approximately 11.5 mM. The combination of the two enzymes further increased the accumulation to 13 mM of butyrate.

Table 2. Mean value of the amount of acetate, propionic acid and butyrate after four cycles of 12-h fermentation of RSM by chicken caecal microbiota with no addition of enzyme (Control), with the addition of 20 ppm of RG-1 lyase (RGL_1), with the addition of 20 ppm of galactanase (GH53), and with a combination of the two enzymes at 20 ppm each. The values are given in mM.

| Treatment | Acetate | Propionic acid | Butyrate |
|---|---|---|---|
| Control | 49.234 | 25.3547 | 8.88528 |
| Galactanase | 52.0745 | 25.688 | 11.3802 |
| RG-I lyase | 56.3005 | 32.0729 | 11.6395 |
| RG-I lyase | 56.8993 | 30.9499 | 13.0665 |
| and Galactanase | | | |

### Example 3: The Effect of Xyloglucan-Specific β-Glucanases on RSM

The effect of two xyloglucan-specific β-glucanases (GH12_1), one from A. aculeatus and one from A. luchuensis, for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase®, the enzymes were inactivated at 80 °C for 15 min. After centrifugation at 3,000 rpm at 0 °C for 15 min, the ethanol from the pellet was evaporated overnight in fume hood and the samples were freeze-dried.

Deproteinized RSM (3% dry matter) was incubated separately with two xyloglucan-specific β-glucanases (GH12_1) in acetate buffer at pH 5.0 for 4 hours at 40 °C. Experiments were run in triplicates.

Following incubations with the enzymes, the solids were removed by centrifugation for 15 min at 4,700 g and 0 °C and 1,000 μL of the supernatant was subjected to acid hydrolysis (0.5 M H₂SO₄) for 50 min at 124 °C in the autoclave. The hydrolysate was filtered through 0.2 μm wwPTFE membrane (AcroPrep Advance 8582, PALL) and analyzed for its monosaccharide contents by high performance anion exchange chromatography combined with pulsed amperometric detection (HPAEC-PAD).

Monosaccharide separation was achieved on a CarboPac analytical PA1 column (4 mm×250 mm) with a CarboPac PA1 guard column (4 mm×50 mm) (Thermofisher) at a temperature of 30 °C with an eluent flow rate fixed at 1 mL/min. The elution of neutral monosaccharides was achieved with 10 mM NaOH as eluent within 14 min. In the sequence, the eluent concentration was increased to 500 mM and the acid monosaccharides were resolved within 7.5 min. The column was re-equilibrated with NaOH 10 mM for 10.5 min before injection of subsequent sample. The concentration of monosaccharides was calculated based on the standard curves of fucose, arabinose, rhamnose, galactose, glucose, xylose, mannose, galacturonic acid and glucuronic acid.

The GH12_1 from A. luchuensis increased the amount of fucose, xylose and glucose in the supernatant of RSM by 4-fold, 45% and 21%, respectively.

The GH12_1 from A. aculeatus increased the amount of fucose, xylose and glucose in the supernatant of RSM by 5-fold, 73% and 35%, respectively.

Table 3. Mean value of the amount of fucose, rhamnose, arabinose, galactose, glucose and xylose after treatment with no addition of enzyme (Control), with the addition of 20 ppm of GH12_1 (A. luchuensis) and with the addition of 20 ppm of GH12_1 (A. aculeatus). The values are given in % of the monosaccharide in rapeseed meal (RSM).

| Treatment | Fucose | Rhamnose | Arabinose | Galactose | Glucose | Xylose |
|---|---|---|---|---|---|---|
| Control | 0.011383 | 0.040478 | 1.228275 | 0.846672 | 1.297418 | 0.374021 |
| GH12_1 / A. luchuensis | 0.041984 | 0.046149 | 1.293083 | 0.962642 | 1.575253 | 0.54325 |
| GH12_1 / A. aculeatus | 0.054315 | 0.053886 | 1.41049 | 1.061931 | 1.75221 | 0.64836 |

### Example 4: The Effect of an Endo-Polygalacturonase on RSM

The effect of an endo-polygalacturonase (GH28_9, A. aculeatus) for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase®, the enzymes were inactivated at 80 °C for 15 min. After centrifugation at 3,000 rpm at 0 °C for 15 min, the ethanol from the pellet was evaporated overnight in fume hood and the samples were freeze-dried.

Deproteinized RSM (3% dry matter) was incubated with an endo-polygalacturonase (GH28_9) in acetate buffer at pH 5.0 for 4 hours at 40 °C. Experiments were run in triplicates.

Following incubations with the enzymes, the solids were removed by centrifugation for 15 min at 4,700 g and 0 °C and 1,000 μL of the supernatant was subjected to acid hydrolysis (0.5 M H₂SO₄) for 50 min at 124 °C in the autoclave. The hydrolysate was filtered through 0.2 μm wwPTFE membrane (AcroPrep Advance 8582, PALL) and analyzed for its monosaccharide contents by high performance anion exchange chromatography combined with pulsed amperometric detection (HPAEC-PAD).

Monosaccharide separation was achieved on a CarboPac analytical PA1 column (4 mm×250 mm) with a CarboPac PA1 guard column (4 mm×50 mm) (Thermofisher) at a temperature of 30 °C with an eluent flow rate fixed at 1 mL/min. The elution of neutral monosaccharides was achieved with 10 mM NaOH as eluent within 14 min. In the sequence, the eluent concentration was increased to 500 mM and the acid monosaccharides were resolved within 7.5 min. The column was re-equilibrated with NaOH 10 mM for 10.5 min before injection of subsequent sample. The concentration of monosaccharides was calculated based on the standard curves of fucose, arabinose, rhamnose, galactose, glucose, xylose, mannose, galacturonic acid and glucuronic acid.

The treatment with an endo-polygalacturonase (GH28_9) increased the amount of monosaccharides in the supernatant of RSM by 14%. The amount of fucose and rhamnose in the soluble fraction of enzymatically treated samples compared to the control was 70% higher, while arabinose, galactose, glucose and xylose solubilization lies between 11 and 17%.

Table 4. Mean value of the amount of fucose, rhamnose, arabinose, galactose, glucose, xylose and the total amount of monosaccharides after treatment with no addition of enzyme (Control) and with the addition of 20 ppm of an endo-polygalacturonase (GH28_9).

The values are given in % of the monosaccharide in rapeseed meal (RSM).

| Treatment | Fucose | Rhamnose | Arabinose | Galactose | Glucose | Xylose | Total |
|---|---|---|---|---|---|---|---|
| Control | 0.01604 | 0.0418 | 1.26044 | 0.80964 | 1.26933 | 0.46345 | 3.8607 |
| Polygalacturonase | 0.02728 | 0.07286 | 1.40152 | 0.92288 | 1.42433 | 0.54479 | 4.39366 |

### Example 5: The Effect of Pectinases on RSM

The effect of two pectinases, one from A. tubingensis and one from A. aculeatus, for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase®, the enzymes were inactivated at 80 °C for 15 min. After centrifugation at 3,000 rpm at 0 °C for 15 min, the ethanol from the pellet was evaporated in fume hood overnight and the samples were freeze-dried.

Deproteinized RSM (3% dry matter) was incubated separately with two pectinases in acetate buffer at pH 5.0 for 4 hours at 40 °C. Experiments were run in triplicates.

Following incubations with the enzymes, the solids were removed by centrifugation for 15 min at 4,700 g and 0 °C and 1,000 μL of the supernatant was subjected to acid hydrolysis (0.5 M H₂SO₄) for 50 min at 124 °C in the autoclave. The hydrolysate was filtered through 0.2 μm wwPTFE membrane (AcroPrep Advance 8582, PALL) and analyzed for its monosaccharide contents by high performance anion exchange chromatography combined with pulsed amperometric detection (HPAEC-PAD).

Monosaccharide separation was achieved on a CarboPac analytical PA1 column (4 mm×250 mm) with a CarboPac PA1 guard column (4 mm×50 mm) (Thermofisher) at a temperature of 30 °C with an eluent flow rate fixed at 1 mL/min. The elution of neutral monosaccharides was achieved with 10 mM NaOH as eluent within 14 min. In the sequence, the eluent concentration was increased to 500 mM and the acid monosaccharides were resolved within 7.5 min. The column was re-equilibrated with NaOH 10 mM for 10.5 min before injection of subsequent sample. The concentration of monosaccharides was calculated based on the standard curves of fucose, arabinose, rhamnose, galactose, glucose, xylose, mannose, galacturonic acid and glucuronic acid.

The pectinase from A. tubingensis increased the amount of xylose and rhamnose in the supernatant of RSM by 10% and 35%, respectively.

The pectinase from A. aculeatus increased the amount of xylose and rhamnose in the supernatant of RSM by 13% and 74%, respectively.

Table 5. Mean value of the amount of fucose, rhamnose, arabinose, xylose and the total amount of monosaccharides after treatment with no addition of enzyme (Control), with the addition of 20 ppm of pectinase (A. tubingensis) and with the addition of 20 ppm of pectinase (A. aculeatus). The values are given in % of the monosaccharide in rapeseed meal (RSM).

| Treatment | Fucose | Rhamnose | Arabinose | Xylose | Total |
|---|---|---|---|---|---|
| Control | 0.01604 | 0.0418 | 1.26044 | 0.46345 | 3.8607 |
| pectinase/ | 0.01824 | 0.07255 | 1.30973 | 0.52326 | 3.93309 |
| A. aculeatus | | | | | |
| pectinase/ | 0.01823 | 0.05638 | 1.31123 | 0.5046 | 4.00899 |
| A. tubingensis | | | | | |

### Example 6: Effect of a Rhamnogalacturonan Endolyase, an Endo-β-1,4-Galactanase and the Combination on Soybean Meal

The effect of a rhamnogalacturonan endolyase (RGL_1, P. oxalicum) and an endo-β-1,4-galactanase (GH53) for application in the animal feed on deproteinized soybean meal (SBM) was investigated in an in vitro experiment.

SBM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase® the enzymes were inactivated at 80 °C for 15 min. After centrifugation at 3,000 rpm at 0 °C for 15 min, the ethanol from the pellets was evaporated overnight in fume hood and the samples were freeze-dried.

Deproteinized SBM (3% dry matter) was incubated with a rhamnogalacturonan endolyase (RGL_1), an endo-β-1,4-galactanase (GH53), or the combination thereof in acetate buffer at pH 5.0 for 4 hours at 40 °C. Experiments were run in triplicates.

Following incubations with the enzymes, the solids were removed by centrifugation for 15 min at 4,700 g and 0 °C and 1,000 μL of the supernatant was subjected to acid hydrolysis (0.5 M H₂SO₄) for 50 min at 124 °C in the autoclave. The hydrolysate was filtered through 0.2 μm wwPTFE membrane (AcroPrep Advance 8582, PALL) and analyzed for its monosaccharide contents by high performance anion exchange chromatography combined with pulsed amperometric detection (HPAEC-PAD).

Monosaccharide separation was achieved on a CarboPac analytical PA1 column (4 mm×250 mm) with a CarboPac PA1 guard column (4 mm×50 mm) (Thermofisher) at a temperature of 30 °C with an eluent flow rate fixed at 1 mL/min. The elution of neutral monosaccharides was achieved with 10 mM NaOH as eluent within 14 min. In the sequence, the eluent concentration was increased to 500 mM and the acid monosaccharides were resolved within 7.5 min. The column was re-equilibrated with NaOH 10 mM for 10.5 min before injection of subsequent sample. The concentration of monosaccharides was calculated based on the standard curves of fucose, arabinose, rhamnose, galactose, glucose, xylose, mannose, galacturonic acid and glucuronic acid.

The experiments demonstrated that approximately 20% more polysaccharides from SBM were solubilized when the RGL_1 is combined with the GH53, compared to the sum of the effect of the two enzymes separately, illustrating the synergy between the two enzyme products. The combination of the two enzymes resulted in 11.3% of the insoluble NSP to be solubilized, while the RGL_1 and the GH53 separately resulted in a solubilization of 1.0% and 8.4% of the insoluble NSP respectively. The main monosaccharides present in the solubilized NSP are rhamnose, galacturonic acid, arabinose, galactose, xylose and fucose.

Table 6. Mean value of the amount of fucose, rhamnose, arabinose, galactose, xylose, galacturonic acid and the total amount of monosaccharides after treatment with no addition of enzyme (Control), with the addition of 20 ppm of RG-I lyase (RGL_1), with the addition of 20 ppm of galactanase (GH53), and with a combination of the two enzymes at 20 ppm each. The values are given in % of the monosaccharide in soybean meal (SBM).

| | | | | | | Galacturonic | |
|---|---|---|---|---|---|---|---|
| Treatment | Fucose | Rhamnose | Arabinose | Galactose | Xylose | acid | Total |
| Control | 0.047 | 0.048 | 0.590 | 2.893 | 0.084 | 0.112 | 9.237 |
| RG-I lyase | 0.046 | 0.166 | 0.799 | 3.473 | 0.082 | 0.193 | 10.300 |
| Galactanase | 0.052 | 0.053 | 1.409 | 10.790 | 0.109 | 0.142 | 17.629 |
| RG-I lyase + | 0.056 | 0.426 | 3.048 | 11.282 | 0.117 | 0.380 | 20.536 |
| Galactanase | | | | | | | |

### Example 7: Effect of a Rhamnogalacturonan Endolyase, an Endo-β-1,4-Galactanase and the Combination on Rapeseed Meal

The effect of a rhamnogalacturonan endolyase (RGL_1, P. oxalicum) and an endo-β-1,4-galactanase (GH53) for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase® the enzymes were inactivated at 80 °C for 15 min. After centrifugation at 3,000 rpm at 0 °C for 15 min, the ethanol from the pellets was evaporated overnight in fume hood and the samples were freeze-dried.

Deproteinized RSM (3% dry matter) was incubated with a rhamnogalacturonan endolyase (RGL_1), an endo-β-1,4-galactanase (GH53), or the combination thereof in acetate buffer at pH 5.0 for 4 hours at 40 °C. Experiments were run in triplicates.

Following incubations with the enzymes, the solids were removed by centrifugation for 15 min at 4,700 g and 0 °C and 1,000 μL of the supernatant was subjected to acid hydrolysis (0.5 M H₂SO₄) for 50 min at 124 °C in the autoclave. The hydrolysate was filtered through 0.2 μm wwPTFE membrane (AcroPrep Advance 8582, PALL) and analyzed for its monosaccharide contents by high performance anion exchange chromatography combined with pulsed amperometric detection (HPAEC-PAD).

Monosaccharide separation was achieved on a CarboPac analytical PA1 column (4 mm×250 mm) with a CarboPac PA1 guard column (4 mm×50 mm) (Thermofisher) at a temperature of 30 °C with an eluent flow rate fixed at 1 mL/min. The elution of neutral monosaccharides was achieved with 10 mM NaOH as eluent within 14 min. In the sequence, the eluent concentration was increased to 500 mM and the acid monosaccharides were resolved within 7.5 min. The column was re-equilibrated with NaOH 10 mM for 10.5 min before injection of subsequent sample. The concentration of monosaccharides was calculated based on the standard curves of fucose, arabinose, rhamnose, galactose, glucose, xylose, mannose, galacturonic acid and glucuronic acid.

The experiments demonstrated that approximately 44% more polysaccharides from RSM were solubilized when the RGL_1 is combined with the GH53, compared to the sum of the effect of the two enzymes separately, illustrating the synergy between the two enzyme products. The combination of the two enzymes resulted in 3.9% of the insoluble NSP to be solubilized, while the RGL_1 and the GH53 separately resulted in a solubilization of 0.5% and 2.2% of the insoluble NSP respectively. The main monosaccharides present in the solubilized NSP are rhamnose, galacturonic acid, arabinose, galactose, and xylose.

Table 7. Mean value of the amount of rhamnose, arabinose, galactose, xylose, galacturonic acid and the total amount of monosaccharides after treatment with no addition of enzyme (Control), with the addition of 20 ppm of RG-I lyase (RGL_1), with the addition of 20 ppm of galactanase (GH53), and with a combination of the two enzymes at 20 ppm each. The values are given in % of the monosaccharide in rapeseed meal (RSM).

| | | | | | Galactu- | |
|---|---|---|---|---|---|---|
| | | | | | ronic | |
| Treatment | Rhamnose | Arabinose | Galactose | Xylose | acid | Total |
| Control | 0.053 | 1.579 | 0.972 | 0.171 | 0.107 | 5.091 |
| RG-I lyase | 0.211 | 2.225 | 1.128 | 0.139 | 0.170 | 5.629 |
| Galactanase | 0.060 | 1.878 | 2.352 | 0.206 | 0.127 | 7.294 |
| RG-I | 0.277 | 3.136 | 2.476 | 0.232 | 0.263 | 9.043 |
| lyase + | | | | | | |
| Galactanase | | | | | | |

### Example 8: Effect of Rhamnogalacturonan Endolyases, an Endo-β-1,4-Galactanase, a Xyloglucanase, and their Combination with a Pectin Lyase on Rapeseed Meal

The effect of two rhamnogalacturonan endolyases (RGL_1, one from A. aculeatus and one from P. oxalicum), an endo-β-1,4-galactanase (GH53), an endo-xyloglucanase (GH5_4), and their combination with a pectin lyase (LYA1_4) for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase® the enzymes were inactivated at 80 °C for 15 min. After centrifugation at 3,000 rpm at 0 °C for 15 min, the ethanol from the pellets was evaporated overnight in fume hood and the samples were freeze-dried.

Deproteinized RSM (3% dry matter) was incubated with a rhamnogalacturonan endolyase (RGL_1), an endo-β-1,4-galactanase (GH53), a xyloglucanase, a pectin lyase and their combination in acetate buffer at pH 5.0 for 4 hours at 40 °C. Experiments were run in triplicates.

Following incubations with the enzymes, the solids were removed by centrifugation for 15 min at 4,700 g and 0 °C and 1,000 μL of the supernatant was subjected to acid hydrolysis (0.5 M H₂SO₄) for 50 min at 124 °C in the autoclave. The hydrolysate was filtered through 0.2 μm wwPTFE membrane (AcroPrep Advance 8582, PALL) and analyzed for its monosaccharide contents by high performance anion exchange chromatography combined with pulsed amperometric detection (HPAEC-PAD).

Monosaccharide separation was achieved on a CarboPac analytical PA1 column (4 mm×250 mm) with a CarboPac PA1 guard column (4 mm×50 mm) (Thermofisher) at a temperature of 30 °C with an eluent flow rate fixed at 1 mL/min. The elution of neutral monosaccharides was achieved with 10 mM NaOH as eluent within 14 min. In the sequence, the eluent concentration was increased to 500 mM and the acid monosaccharides were resolved within 7.5 min. The column was re-equilibrated with NaOH 10 mM for 10.5 min before injection of subsequent sample. The concentration of monosaccharides was calculated based on the standard curves of fucose, arabinose, rhamnose, galactose, glucose, xylose, mannose, galacturonic acid and glucuronic acid.

The experiments demonstrated that combining the pectin lyase with one of the other enzymes doubled the amount of total monosaccharides in the hydrolysate of the soluble fraction of RSM compared to the control.

The total amount of monosaccharides (mg) in the soluble fraction of 100 mg of RSM is increased by more than 5 percentage points (pp) when the xyloglucanase is combined with the pectin lyase, compared to the sum of the effect of these two enzymes separately, illustrating the synergy between the two enzymes. The combination of the xyloglucanase and the pectin lyase enzymes improved the solubilization of fucose-, rhamnose-, arabinose-, galactose-, glucose-, xylose- and galacturonic acid-containing polysaccharides. The main improvements in percentage of soluble monosaccharide per total plant material were observed for arabinose-, galactose-, xylose- and galacturonic acid-containing polysaccharides, with 1.5 pp, 0.8 pp, 0.6 pp and 0.3 pp increase in the soluble fraction of RSM treated with the combined enzymes compared to the sum of the treatments with the two enzymes separately.

The total amount of monosaccharides (mg) in the soluble fraction of 100 mg of RSM is increased by approximately 3 pp when the galactanase is combined with the pectin lyase, compared to the sum of the effect of these two enzymes separately, illustrating the synergy between the two enzymes. The combination of the galactanase and the pectin lyase enzymes improved the solubilization of fucose-, rhamnose-, arabinose-, galactose-, glucose-, xylose- and galacturonic acid-containing polysaccharides. The main improvements in percentage of soluble monosaccharide per total plant material were observed for arabinose-, galactose-, xylose- and galacturonic acid-containing polysaccharides, with 1.1 pp, 0.2 pp, 0.3 pp and 0.3 pp increase in the soluble fraction of RSM treated with the combined enzymes compared to the sum of the treatments with the two enzymes separately.

The total amount of monosaccharides (mg) in the soluble fraction of 100 mg of RSM is increased by more than 2 pp when the RG-1 lyase (A. aculeatus) is combined with the pectin lyase, compared to the sum of the effect of these two enzymes separately, illustrating the synergy between the two enzymes. The combination of the RG-1 lyase (A. aculeatus) and the pectin lyase enzymes improved the solubilization of fucose-, rhamnose-, arabinose-, galactose-, glucose-, xylose- and galacturonic acid-containing polysaccharides. The main improvements in percentage of soluble monosaccharide per total plant material were observed for arabinose-, galactose-, xylose- and galacturonic acid-containing polysaccharides, with 0.9 pp, 0.4 pp, 0.2 pp and 0.1 pp increase in the soluble fraction of RSM treated with the combined enzymes compared to the sum of the treatments with the two enzymes separately.

The total amount of monosaccharides (mg) in the soluble fraction of 100 mg of RSM is increased by approximately 5 pp when the RG-1 lyase (P. oxalicum) is combined with the pectin lyase, compared to the sum of the effect of these two enzymes separately, illustrating the synergy between the two enzymes. The combination of the RG-1 lyase (P. oxalicum) and the pectin lyase enzymes improved the solubilization of fucose-, rhamnose-, arabinose-, galactose-, glucose-, xylose- and galacturonic acid-containing polysaccharides. The main improvements in percentage of soluble monosaccharide per total plant material were observed for arabinose-, galactose-, xylose- and galacturonic acid-containing polysaccharides, with 1.6 pp, 0.7 pp, 0.4 pp and 0.4 pp increase in the soluble fraction of RSM treated with the combined enzymes compared to the sum of the treatments with the two enzymes separately.

Table 8. Mean value of the amount of fucose, rhamnose, arabinose, galactose, xylose, galacturonic acid and the total amount of monosaccharides after treatment with no addition of enzyme (Control), with the addition of 20 ppm of pectin lyase (LYA1_4), with the addition of 20 ppm of xyloglucanase (GH5_4), galactanase (GH53), RG-1 lyases (RGL_1) from A. aculeatus and from P. oxalicum, and their combination with the pectin lyase at 20 ppm each. The values are given in % of the monosaccharide in rapeseed meal (RSM).

| | | | | | | Galacturonic | |
|---|---|---|---|---|---|---|---|
| Treatment | Fucose | Rhamnose | Arabinose | Galactose | Xylose | acid | Total |
| Control | 0.02429 | 0.05343 | 1.57905 | 0.97235 | 0.17064 | 0.10678 | 5.091 |
| Pectin lyase | 0.02133 | 0.18066 | 1.72986 | 0.86659 | 0.51743 | 0.47644 | 5.25324 |
| Xyloglucanase | 0.03754 | 0.04637 | 1.28572 | 0.87293 | 0.21679 | 0.07187 | 4.40638 |
| Xyloglucanase + | 0.14194 | \|0.3181 | 3.20382 | 1.82839 | 1.15878 | 0.77494 | 10.7727 |
| Pectin lyase | | | | | | | |
| Galactanase | 0.02929 | 0.05992 | 1.87801 | 2.35208 | 0.20638 | 0.12661 | 7.29426 |
| Galactanase + | 0.05863 | 0.31645 | 3.15283 | 2.56953 | 0.86987 | 0.83911 | 10.4904 |
| Pectin lyase | | | | | | | |
| RG-I lyase (A. aculeatus) | 0.02949 | 0.15604 | 2.24679 | 1.25651 | 0.18595 | 0.18916 | 6.66737 |
| RG-I lyase (A. aculeatus) + | 0.05016 | 0.37592 | 3.28485 | 1.6163 | 0.76227 | 0.71166 | 9.18743 |
| Pectin lyase | | | | | | | |
| RG-I lyase (P. oxalicum) | 0.01568 | 0.21066 | 2.22479 | 1.12794 | 0.13945 | 0.16997 | 5.62873 |
| RG-I lyase (P. oxalicum) + | 0.05346 | 0.47649 | 3.9862 | 1.84106 | 0.88798 | 0.90114 | 10.784 |
| Pectin lyase | | | | | | | |

### Example 9: Effect of Rhamnogalacturonan Endolyases, an Endo-β-1,4-Galactanase, and their Combination with a Pectin Lyase on the Accumulation of Short-Chain Fatty Acids in In Vitro Fermentation of Rapeseed Meal by Chicken Caecal Microbiota

The effect of two rhamnogalacturonan endolyases (RGL_1, one from A. aculeatus and one from P. oxalicum), an endo-β-1,4-galactanase (GH53), and their combination with a pectin lyase (LYA1_4) for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase® (Novozymes A/S) for 3 hours at 50 °C followed by precipitation in ethanol 80%. After the second incubation with Alcalase®, the enzymes were inactivated at 80 °C for 15 min. After centrifugation at 3,000 rpm at 0 °C for 15 min, the ethanol from the pellet was evaporated overnight in fume hood and the samples were freeze-dried.

Deproteinized RSM (3% dry matter) with and without rhamnogalacturonan endolyases (20 ppm), endo-β-1,4-galactanase (20 ppm), and their combination with a pectin lyase (20 ppm) were diluted in anoxic sterile YCFA medium prepared as described by Duncan et al (Duncan, S. H., Hold, G. L., Barcenilla, A., Stewart, C. S. & Flint, H. J. Roseburia intestinalis sp. nov., a novel saccharolytic, butyrate-producing bacterium from human faeces. Int. J. Syst. Evol. Microbiol. 52, 1615-1620 (2002)) modified so the carbon source is 1% of non-starch polysaccharides from deproteinized RSM and the pH was adjusted to 6.5. A pool of caecal content from four 35-day-old broilers was added to a final 1,000-fold dilution for the first round of fermentation. After 12 hours of fermentation at 37 °C, the first fermentation was used as inoculum for the second fermentation and so on. Fermentation supernatants were sampled after 12 hours and stored at −20 °C until analysis. Fermentations were run in five replicates.

The quantification of acetate, propionic acid and butyrate in the supernatants was achieved by running the samples in a high-performance liquid chromatography set-up equipped with an ion-exchange BioRad HPX-87H column with a BioRad Cation H precolumn at 60 °C and a refractive index detector.

The experiment demonstrates that the treatment with rhamnogalacturonan endolyase (A. aculeatus), the rhamnogalacturonan endolyase (P. oxalicum), the galactanase and the pectin lyase separately increased the accumulation of butyrate by chicken caecal microbiota fermentation of RSM from 2.34 mM to 4.71 mM, 2.99 mM, 7.09 mM and 5.4 mM respectively. The combination of the rhamnogalacturonan endolyase (A. aculeatus) with the pectin lyase further increased the accumulation to 7.91 mM of butyrate. The combination of the rhamnogalacturonan endolyase (P. oxalicum) with the pectin lyase further increased the accumulation to 8.64 mM of butyrate. The combination of the galactanase with the pectin lyase further increased the accumulation to 9.63 mM of butyrate.

The experiment demonstrates that the treatment with rhamnogalacturonan endolyase (A. aculeatus), the rhamnogalacturonan endolyase (P. oxalicum), the galactanase and the pectin lyase separately increased the accumulation of acetate by chicken caecal microbiota fermentation of RSM from 71.8 mM to 86.1 mM, 85.2 mM, 88.3 mM and 81.8 mM respectively. The combination of the rhamnogalacturonan endolyase (A. aculeatus) with the pectin lyase further increased the accumulation to 91.3 mM of acetate. The combination of the rhamnogalacturonan endolyase (P. oxalicum) with the pectin lyase further increased the accumulation to 94.6 mM of acetate. The combination of the galactanase with the pectin lyase further increased the accumulation to 96.9 mM of acetate.

The experiment demonstrates that the treatment with rhamnogalacturonan endolyase (A. aculeatus), the rhamnogalacturonan endolyase (P. oxalicum), the galactanase and the pectin lyase separately increased the accumulation of propionate by chicken caecal microbiota fermentation of RSM from 15.3 mM to 19.8 mM, 17.5 mM, 17.5 mM and 20.5 mM respectively. The combination of the rhamnogalacturonan endolyase (A. aculeatus) with the pectin lyase further increased the accumulation to 24.3 mM of propionate. The combination of the rhamnogalacturonan endolyase (P. oxalicum) with the pectin lyase further increased the accumulation to 27.9 mM of propionate. The combination of the galactanase with the pectin lyase further increased the accumulation to 26.1 mM of propionate.

Table 9. Mean value of the amount of acetate, butyrate and propionate after four cycles of 12-h fermentation of RSM by chicken caecal microbiota with no addition of enzyme (Control), with the addition of 20 ppm of RG-1 lyase from A. aculeatus (RGL_1), 20 ppm of RG-1 lyase from A. aculeatus (RGL_1), 20 ppm of galactanase (GH53), 20 ppm of pectin lyase, and with a combination of the pectin lyase with the other three enzymes at 20 ppm each. The values are given in mM.

| Enzyme treatment | Acetate | Butyrate | Propionate |
|---|---|---|---|
| Control | 71.77 | 2.34 | 15.27 |
| RG-I lyase (A. aculeatus) | 86.11 | 4.71 | 19.85 |
| Galactanase | 88.28 | 7.09 | 17.55 |
| Pectin lyase | 81.77 | 5.40 | 20.55 |
| RG-I lyase (P. oxalicum) | 85.19 | 2.99 | 17.45 |
| RG-I lyase (A. aculeatus) + Pectin lyase | 91.34 | 7.91 | 24.28 |
| Galactanase + Pectin lyase | 96.89 | 9.63 | 26.09 |
| RG-I lyase (P. oxalicum) + Pectin lyase | 94.56 | 8.64 | 27.90 |

### Example 10: The Effect of an Endo-β-1,4-Galactanase, and the Combination with a Rhamnogalacturonan Endolyase on the Taxonomic Distribution of Chicken Caecal Microbiota after In Vitro Fermentation of Rapeseed Meal

The effect of a rhamnogalacturonan endolyase (RGL_1, A. aculeatus) and an endo-β-1,4-galactanase (GH53) for application in the animal feed on deproteinized rapeseed meal (RSM) was investigated in an in vitro experiment.

RSM sample procured from DSM Nutritional Products (Village-Neuf, France) was deproteinized through double incubations with a protease Alcalase®(Novozymes A/S) for 3 hours at 50 °C followed by precipitation in ethanol 80%. After the second incubation with Alcalase®, the enzymes were inactivated at 80 °C for 15 min. After centrifugation at 3,000 rpm at 0 °C for 15 min, the ethanol from the pellet was evaporated overnight in fume hood and the samples were freeze-dried.

Deproteinized RSM (3% dry matter) without any enzymes, with an endo-β-1,4-galactanase (20 ppm) and with an endo-β-1,4-galactanase (20 ppm) combined with a rhamnogalacturonan endolyase were diluted in anoxic sterile YCFA medium prepared as described by Duncan et al (Duncan, S. H., Hold, G. L., Barcenilla, A., Stewart, C. S. & Flint, H. J. Roseburia intestinalis sp. nov., a novel saccharolytic, butyrate-producing bacterium from human faeces. Int. J. Syst. Evol. Microbiol. 52, 1615-1620 (2002)) modified so the carbon source was 1% of non-starch polysaccharides from deproteinized RSM and the pH was adjusted to 6.5. Chicken caecal content from 35-day-old broiler was added to a final 1,000-fold dilution for the first round of fermentation. Fermentations were run in five replicates. After 12 hours of fermentation at 37 °C fermentations were sampled and stored at −20 °C until analysis. The DNA was extracted following the DNeasy Ultra Clean Microbial Kit (Qiagen, January 2020) protocol.

For library preparation, the 16S rRNA gene amplicons were prepared for sequencing in the Illumina MiSeq System according to the protocol provided by Illumina (support document, part 15044223, rev. B) targeting the V3-V4 regions of 16S.

The experiment demonstrates that the abundance of Enterococcus and Escherichia/Shigella in the fermentation of RSM by chicken caecal microbiota dropped in the presence of enzymes, both in the presence of galactanase (20 ppm) and its combination with the RG-I lyase (20 ppm).

The enzymatic treatments favoured the growth of Bacteroides, a genus that contains well known pectin-degrading bacteria that, in a cross-feeding manner, benefits the growth of butyrate producers. In the presence of the galactanase, the relative abundance of Bacteroides increased 5%, and, when combined with the RG-I lyase, the relative abundance increased 10% compared to the Control treatment.

Propionibacterium, genus known for its propionic acid producers, had its relative abundance doubled when the RG-1 lyase was added to the galactanase, compared to the galactanase alone.

The genus Lactobacillus, which hosts the most common probiotics, had its relative abundance increased from 1.9% to 2.7% when RSM was treated with galactanase, and further increased to 4.5% when the RG-1 lyase was combined with the galactanase.

The relative abundance of the genera Butyricicoccus increased 40% in the presence of the galactanase, and it nearly tripled when combined with the RG-1 lyase, compared to the Control treatment. This genus contains butyrate producers.

Table 10. Relative abundances of the top ten bacteria identified using sequencing of V3-V4 regions of 16S rRNA after the fermentation of RSM by chicken caecal microbiota with no addition of enzyme (Control), with the addition of 20 ppm of galactanase (GH53), and with the combination with 20 ppm of RG-1 lyase (RGL_1). The values are given in percentage.

| | RSM | RSM | RSM (galactanase + |
|---|---|---|---|
| Genus | (Control) | (galactanase) | RG-I lyase) |
| Enterococcus | 42.4 | 38.4 | 33.7 |
| Escherichia/Shigella | 11.1 | 10.3 | 8.6 |
| Bacteroides | 23.0 | 24.2 | 25.3 |
| Streptococcus | 4.1 | 4.5 | 7.2 |
| Faecalibacterium | 4.4 | 4.8 | 4.5 |
| Clostridium_XVIII | 1.7 | 1.6 | 1.4 |
| Clostridium_XIVb | 0.8 | 0.9 | 1.4 |
| Propionibacterium | 2.2 | 2.6 | 5.1 |
| Lactobacillus | 1.9 | 2.7 | 4.5 |
| Butyricicoccus | 0.5 | 0.7 | 1.4 |

### Example 11: Animal Feed and Feed Additive Compositions

A formulation of a fiber-degrading enzyme containing 0.050 g enzyme protein is added to the following premix (per kilo of premix):

| | | |
|---|---|---|
| 5000000 | IE | Vitamin A |
| 1000000 | IE | Vitamin D3 |
| 13333 | mg | Vitamin E |
| 1000 | mg | Vitamin K3 |
| 750 | mg | Vitamin B1 |
| 2500 | mg | Vitamin B2 |
| 1500 | mg | Vitamin B6 |
| 7666 | mcg | Vitamin B12 |
| 12333 | mg | Niacin |
| 33333 | mcg | Biotin |
| 300 | mg | Folic Acid |
| 3000 | mg | Ca-D-Panthothenate |
| 1666 | mg | Cu |
| 16666 | | mg Fe |
| 16666 | mg | Zn |
| 23333 | mg | Mn |
| 133 | mg | Co |
| 66 | mg | I |
| 66 | mg | Se |
| 5.8 | % | Calcium |
| 25 | % | Sodium |

### Animal Feed

This is an example of an animal feed (broiler feed) comprising 0.5 mg/kg (0.5 ppm) of the fiber-degrading enzyme (calculated as pectinase enzyme protein):
65.00% wheat
32.35% Soybean meal (50% crude protein, CP)
1.0% Soybean oil
0.2% DL-Methionine
0.22% DCP (dicalcium phosphate)
0.76% CaCO₃ (calcium carbonate)
0.32% Sand
0.15% NaCl (sodium chloride)
1% of the above Premix

The ingredients are mixed, and the feed is pelleted at the desired temperature, e.g., 70 °C.

### Example 12: Cloning of GH53 Galactanases from Cohnella sp-60555 (Mature Polypeptide of SEQ ID NO: 5 with His-Tag)

The genes encoding the galactanases were amplified by PCR and fused with regulatory elements, affinity purification tag and homology regions for recombination into the B. subtilis genome. The linear integration construct was a SOE-PCR fusion product (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68) made by fusion of the gene between two Bacillus subtilis chromosomal regions along with strong promoters and a chloramphenicol resistance marker. The SOE PCR method is also described in patent application WO 2003095658.

The gene was expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence.

The gene was expressed with a Bacillus clausii secretion signal (encoding the following amino acid sequence: MKKPLGKIVASTALLISVAFSSSIASA, SEQ ID NO: 11) replacing the native secretion signal. Furthermore, the expression construct results in the addition of an amino terminal poly histidine purification tag on the natural mature protein allowing for enzyme purification through immobilized metal ion affinity chromatography.

The SOE-PCR product was transformed into Bacillus subtilis and integrated in the chromosome by homologous recombination into the pectate lyase locus. Subsequently one recombinant Bacillus subtilis clone containing the respective galactanase expression construct was selected and was cultivated on a rotary shaking table in 500 ml baffled Erlenmeyer flasks each containing 100 ml rich starch based media. After 3-5 days cultivation time at 30 °C to 37 °C, enzyme containing supernatants were harvested by centrifugation and the enzymes were purified by immobilized metal affinity chromatography.

### Example 13: Purification of GH53 Galactanases from Cohnella sp-60555 (Mature Polypeptide of SEQ ID NO: 5 with His-Tag)

The pH of the supernatant from example 12 was adjusted to pH 8, filtrated through a 0.2 μM filter, and then applied to a 5 ml HisTrap™ excel column (GE Healthcare Life Sciences, Pittsburgh, USA). Prior to loading, the column had been equilibrated in 5 column volumes (CV) of 50 mM Tris/HCl pH 8. In order to remove unbound material, the column was washed with 8 CV of 50 mM Tris/HCl pH 8, and elution of the target was obtained with 50 mM HEPES pH 7+10 mM imidazole. The eluted protein was desalted on a HiPrep™ 26/10 desalting column (GE Healthcare Life Sciences, Pittsburgh, USA)., equilibrated using 3 CV of 50 mM HEPES pH 7+100 mM NaCl. This buffer was also used for elution of the target, and the flow rate was 10 ml/min. Relevant fractions were selected and pooled based on the chromatogram and SDS-PAGE analysis.

### Galactanase Assay

Galactanase activity can be determined using the reducing ends colorimetric assay. 10% soybean meal substrate (prepared from soybean meal milled to a 0.5 mm particle size) was filled with a solid dispenser into 96 well format plates. The weight was measured before and after addition of soybean meal and the substrate amount per well was estimated assuming equal distribution along the plate.

The enzymes were diluted to 0.6 ppm (final enzyme concentration in solution) in 100 mM activity buffer (100 mM acetate, 100 mM MES, 100 mM Glycine in 0.01% Triton X100, 1 mM CaCl₂, pH 6.5) and the samples were shaken for 2 hours at 40 °C. The samples were centrifuged at 3000×g for 5 minutes and 75 μl of each sample (supernatant) was transferred to a new PCR-plate. 75 μl activity buffer was added to each sample, the samples were mixed then 75 μl of stop solution (15 mg/ml PAHBAH (Sigma H-9882) in Ka-Na-tartrate/NaOH solution, pH>10) was added. The solution was mixed for 10 min at 95 °C, then 1 min. 10 °C and the samples were transferred to a new 96 MTP and absorbance was measured at 405 nm.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An animal feed comprising an oil seed material and one or more fiber-degrading enzymes.

2. The animal feed of claim 1, wherein said one or more fiber-degrading enzymes comprises at least two fiber-degrading enzymes.

3. The animal feed of claim 1, wherein said one or more fiber-degrading enzymes comprises a rhamnogalacturonan lyase and/or a galactanase and/or a pectin lyase.

4. The animal feed of claim 1, wherein said one or more fiber-degrading enzymes comprises a rhamnogalacturonan lyase and a galactanase.

5. The animal feed of claim 1, comprising oil seed material in an amount of 10 to 500 g/kg feed and the fiber-degrading enzyme in an amount of 0.1 to 500 mg enzyme protein/kg of feed.

6. The animal feed of claim 1, further comprising one or more additional components selected from vitamins, minerals, amino acids, phytogenics, probiotics, organic acids, and other feed ingredients.

7. The animal feed of claim 1, wherein said one or more fiber-degrading enzymes comprises one or more enzymes selected from (alpha-L-rhamnopyranosyl-1,4-alpha-D-galactopyranosyluronate endolyases, endo-beta-1,4-galactanases, 1,4-alpha-D-galacturonan glycanohydrolases, rhamnogalacturonan alpha-D-galacturonic acid-1,2-alpha-L-rhamnose hydrolases, pectin methyl esterases, pectin pectyl hydrolases, (1,4)-6-O-methyl-alpha-D-galacturonan lyases, pectin acetyl esterases, acetic ester acetylhydrolases, galactan endo-beta-1,3-galactanases, xylogalacturonases, and combinations thereof.

8. The animal feed of claim 1, wherein said one or m ore fiber-degrading enzymes comprises:
(a) a rhamnogalacturonan lyase having at least 60% sequence identity to SEQ ID NO: 1 or a mature polypeptide of SEQ ID NO: 1;
(b) a rhamnogalacturonan lyase derived from SEQ ID NO: 1 or a mature polypeptide of SEQ ID NO: 1;
(c) a rhamnogalacturonan lyase derived from (a), or (b), wherein the N- and/or C-terminal end has been extended by addition of one or more amino acids;
(d) a fragment of the rhamnogalacturonan lyase of (a), (b), or (c), having rhamnogalacturonan lyase activity;
(e) an endo-polygalacturonase having at least 60% sequence identity to SEQ ID NO: 2 or a mature polypeptide of SEQ ID NO: 2;
(f) an endo-polygalacturonase derived from SEQ ID NO: 2 or a mature polypeptide of SEQ ID NO: 2;
(g) an endo-polygalacturonase derived from (e), or (f), wherein the N- and/or C-terminal end has been extended by addition of one or more amino acids;
(h) a fragment of the endo-polygalacturonase of (e), (f), or (g), having endo-polygalacturonase activity;
(i) a xyloglucan-specific endo-1,4-beta-glucanase having at least 60% sequence identity to SEQ ID NO: 3 or a mature polypeptide of SEQ ID NO: 3;
(j) a xyloglucan-specific endo-1,4-beta-glucanase derived from SEQ ID NO: 3 or a mature polypeptide of SEQ ID NO: 3;
(k) a xyloglucan-specific endo-1,4-beta-glucanase derived from (i), or (j), wherein the N- and/or C-terminal end has been extended by addition of one or more amino acids;
(l) a fragment of the xyloglucan-specific endo-1,4-beta-glucanase of (i), (j), or (k), having xyloglucan-specific endo-1,4-beta-glucanase activity;
(m) a xyloglucan-specific endo-1,4-beta-glucanase having at least 60% sequence identity to SEQ ID NO: 4 or a mature polypeptide of SEQ ID NO: 4;
(n) a xyloglucan-specific endo-1,4-beta-glucanase derived from SEQ ID NO: 4 or a mature polypeptide of SEQ ID NO: 4;
(o) a xyloglucan-specific endo-1,4-beta-glucanase derived from (m), or (n), wherein the N- and/or C-terminal end has been extended by addition of one or more amino acids; and
(p) a fragment of the xyloglucan-specific endo-1,4-beta-glucanase of (m), (n), or (o), having xyloglucan-specific endo-1,4-beta-glucanase activity;
(q) a galactanase having at least 60% sequence identity to SEQ ID NO: 5 or a mature polypeptide of SEQ ID NO: 5;
(r) a galactanase derived from SEQ ID NO: 5 or a mature polypeptide of SEQ ID NO: 5;
(s) a galactanase derived from (q) or (r), wherein the N- and/or C-terminal end has been extended by addition of one or more amino acids;
(t) a fragment of the galactanase of (q), (r), or (s), having galactanase activity;
(u) a xylogalacturonase having at least 60% sequence identity to SEQ ID NO: 6 or a mature polypeptide of SEQ ID NO: 6;
(v) a xylogalacturonase derived from SEQ ID NO: 6 or a mature polypeptide of SEQ ID NO: 6;
(w) a xylogalacturonase derived from (u) or (v), wherein the N- and/or C-terminal end has been extended by addition of one or more amino acids;
(x) a fragment of the xylogalacturonase of (u), (v), or (w), having xylogalacturonase activity;
(y) a xylogalacturonase having at least 60% sequence identity to SEQ ID NO: 7 or a mature polypeptide of SEQ ID NO: 7;
(z) a xylogalacturonase derived from SEQ ID NO: 7 or a mature polypeptide of SEQ ID NO: 7;
(aa) a xylogalacturonase derived from (y) or (z), wherein the N- and/or C-terminal end has been extended by addition of one or more amino acids;
(bb) a fragment of the xylogalacturonase of (y), (z) or (aa) having xylogalacturonase activity;
(cc) a pectin lyase having at least 600% sequence identity to SEQ ID NO: 8 or a mature polypeptide of SEQ ID NO: 8;
(dd) a pectin lyase derived from SEQ ID NO: 8 or a mature polypeptide of SEQ ID NO: 8;
(ee) a pectin lyase derived from (cc) or (dd), wherein the N- and/or C-terminal end has been extended by addition of one or more amino acids;
(ff) a fragment of the pectin lyase of (cc), (dd), or (ee) having pectin lyase activity;
(gg) a rhamnogalacturonan lyase having at least 60% sequence identity to SEQ ID NO: 9 or a mature polypeptide of SEQ ID NO: 9;
(hh) a rhamnogalacturonan lyase derived from SEQ ID NO: 9 or a mature polypeptide of SEQ ID NO: 9;
(ii) a rhamnogalacturonan lyase derived from (gg) or (hh), wherein the N- and/or C-terminal end has been extended by addition of one or more amino acids; and
(jj) a fragment of the rhamnogalacturonan lyase of (gg), (hh) or (ii) having rhamnogalacturonan lyase activity;
(kk) a xyloglucan-specific endo-b-1,4-glucanase/endo-xyloglucanase having at least 60% sequence identity to SEQ ID NO: 10 or a mature polypeptide of SEQ ID NO: 10;
(ll) a xyloglucan-specific endo-b-1,4-glucanase/endo-xyloglucanase derived from SEQ ID NO: 10 or a mature polypeptide of SEQ ID NO: 10;
(mm) a xyloglucan-specific endo-b-1,4-glucanase/endo-xyloglucanase derived from (kk) or (ll), wherein the N- and/or C-terminal end has been extended by addition of one or more amino acids; and/or
(nn) a fragment of the xyloglucan-specific endo-b-1,4-glucanase/endo-xyloglucanase of (kk), (ll) or (mm) having xyloglucan-specific endo-b-1,4-glucanase/endo-xyloglucanase activity.

9. (canceled)

10. An animal feed additive comprising a polypeptide having rhamno-galacturonan lyase activity and a polypeptide having galactanase activity.
